(19) United States
(12) Patent Application Publication
(10) Pub. No.: US 2012/0265186 A1
(43) Pub. Date: Oct. 18, 2012

(54) STEERABLE CURVABLE ABLATION CATHETER FOR VERTEBROPLASTY

(76) Inventors: Keith Burger, San Francisco, CA (US); John Stalcup, Glen Ellen, CA (US); Michael T. Lyster, Riverwoods, IL (US); Gerard von Hoffmann, Trabucco Canyon, CA (US); Arvind Soni, Lakeland, FL (US)

(21) Appl. No.: 13/219,445

(22) Filed: Aug. 26, 2011

Related U.S. Application Data
(63) Continuation of application No. 12/987,923, filed on Jan. 10, 2011, now abandoned.
Application No. 12/987,923 is a continuation of application No. 12/784,371, filed on May 20, 2010, now abandoned.
(60) Provisional application No. 61/180,058, filed on May 20, 2009.

(51) Int. Cl. A61B 18/14; A61B 18/02; A61B 18/18

(52) U.S. Cl. 606/21; 606/41; 606/33

(57) ABSTRACT

Disclosed herein is a steerable, curvable catheter with one, two, or more ablation elements that can be used for various applications including vertebroplasty. The catheter can include an elongate, tubular body, having a proximal end, a distal end, and a central lumen extending therethrough; a deflectable zone on the distal end of the tubular body, deflectable through an angular range; a handle on the proximal end of the tubular body; and a deflection control on the handle; an energy delivery control on the handle; and one or more energy delivery elements on the distal end of the device for ablating tissue within bone. The energy delivery elements could be RF heating electrodes in a monopole or dipole arrangement although other energy delivery modalities are also contemplated. For example, the energy delivery elements include cryoprobes. Systems and methods involving the ablation catheter are also disclosed.

### PRIORITY CLAIM

This application claims priority under 35 U.S.C. §120 as a continuation application of U.S. patent application Ser. No. 12/987,923 filed Jan. 10, 2011 which is a continuation application of U.S. patent application Ser. No. 12/784,371 filed on May 20, 2010, which in turn claims priority under 35 U.S.C. §119(e) as a nonprovisional of U.S. Provisional App. No. 61/180,058 filed on May 20, 2009. All if the aforementioned priority applications are hereby incorporated by reference in their entireties.

### SUMMARY OF THE INVENTION

In one embodiment of the invention, disclosed is a steerable, curvable energy delivery catheter that can be used in a wide variety of applications including vertebroplasty and kyphoplasty. The catheter can include an elongate, tubular body, having a proximal end, a distal end, and a central lumen extending therethrough; a deflectable zone on the distal end of the tubular body, deflectable through an angular range; a handle on the proximal end of the tubular body; and a deflection control on the handle. The catheter can also include one or two or more energy delivery elements that can be, for example, in the vicinity of the deflectable zone of the device. In some embodiments, the energy delivery elements can be bipolar or monopolar RF electrodes in some embodiments, or as otherwise described in the application. For example, in other embodiments, the energy delivery elements could alternatively or additionally include a cryoprobe. The energy delivery elements can be connected via fiber optics, conductive wires, one or more lumens, other modalities, or wirelessly to an energy source. The energy source can be an RF generator in some embodiments. In other embodiments, the energy source can include a cryogenic source.

In another embodiment, a steerable and curvable ablation catheter is provided. The catheter comprises an elongate tubular body having a proximal end and a distal end, wherein the distal end includes a deflectable zone deflectable through an angular range; a handle on the proximal end of the tubular body; a deflection control on the handle; and an ablation element configured to ablate tissue carried by the deflectable zone. The ablation element can comprise a radiofrequency (RF) electrode or cryoprobe. The steerable and curvable ablation catheter can also comprise an actuator extending axially between the deflection control and the deflectable zone, wherein the actuator comprises and axially moveable element. The deflectable zone can be in a substantially straight configuration from the proximal end to the distal end in an unstressed state. The steerable and curvable catheter can include more than one ablation element.

In another embodiment, a method of ablating tissue is provided. The method comprises positioning a catheter near a zone of tissue to be ablated, the catheter having an elongate body having a proximal end, a deflection control carried by the proximal end, a deflectable distal end, and an ablation element carried by the deflectable distal end; deflecting at least a portion of the distal end of the elongate body through an angular range; and contracting the tissue with the ablation element to ablate the tissue. The ablation element can be an RF heating electrode or a cryoprobe. The tissue to be ablated can comprise cortical bone, cancellous bone, a vertebral body, or a tumor. The ablation can occur as part of a vertebroplasty procedure.

### BRIEF DESCRIPTION OF THE DRAWINGS

FIG. 1 is a perspective view of a steerable injection needle in accordance with one aspect of the present invention.

FIG. 2 is a perspective view of an introducer in accordance with one aspect of the present invention.

FIG. 3 is a perspective view of a stylet in accordance with one aspect of the present invention.

FIG. 4 is a side elevational view of the steerable injection needle moveably coaxially disposed within the introducer, in a substantially linear configuration.

FIG. 5 is a side elevational view of the assembly of FIG. 4, showing the steerable injection needle in a curved configuration.

FIG. 6 is a side elevational schematic view of another steerable injection needle in accordance with the present invention.

FIG. 7A is a schematic view of a distal portion of the steerable needle of FIG. 6, shown in a linear configuration.

FIG. 7B is a schematic view as in FIG. 7A, following proximal retraction of a pull wire to laterally deflect the distal end.

FIG. 8 is a schematic view of a distal portion of a steerable needle, having a side port.

FIG. 9A is a schematic view of a distal portion of a steerable needle, positioned within an outer sheath.

FIG. 9B is an illustration as in FIG. 9A, with the distal sheath partially proximally retracted.

FIG. 9C is an illustration as in FIG. 9B, with the outer sheath proximally retracted a sufficient distance to fully expose the deflection zone.

FIGS. 10A-10C illustrate various aspects of an alternative deflectable needle in accordance with the present invention.

FIGS. 11A through 11C illustrate various aspects of a further deflectable needle design in accordance with the present invention.

FIGS. 12 and 13 illustrate a further variation of the deflectable needle design in accordance with the present invention.

FIG. 14 is a side elevational cross section through the proximal handle of the deflectable needle illustrated in FIG. 13.

FIG. 15 is a cross sectional detail view of the distal tip of the steerable needle illustrated in FIG. 13.

FIGS. 15A through 15H illustrate various views of alternative distal tip designs.

FIGS. 16A and 16B are schematic illustrations of the distal end of a steerable injection device in accordance with the present invention, having a cavity creating element thereon.

FIGS. 16C and 16D are alternative cross sectional views taken along the line 16C-16C in FIG. 16A, showing different inflation lumen configurations.

FIGS. 16E-16G illustrate cross-sections of further alternative inflation lumen configurations.

FIG. 16H schematically illustrates the distal end of a steerable injection device having a cavity creation element with a braided layer.

FIG. 16I illustrates a cross-section through line 16I-16I of FIG. 16H, which some elements omitted for clarity.

FIG. 16J illustrates a cross-section similar to that of FIG. 16I with an additional exterior layer.

FIGS. 16K-16M illustrate various views of an asymmetrical cavity creation element, according to some embodiments of the invention.

FIGS. 16O and 16P schematically illustrate views of a catheter with a plurality of coaxial balloons, according to some embodiments of the invention.

FIGS. 17A and 17B illustrate an alternative steerable injection device having a cavity creation element thereon.

FIG. 17C and FIG. 17D illustrate an alternative steerable injection device having a plurality of cavity creation elements thereon.

FIGS. 17E and 17F are alternative cross sectional views showing different inflation lumen configurations.

FIGS. 17G-17J illustrate further alternative steerable injection devices having a plurality of cavity creation elements thereon.

FIGS. 18A and 18B are schematic views of a bone cement delivery system in accordance with the present invention.

FIGS. 19A through 19F show stages in the method of accomplishing vertebroplasty in accordance with the present invention.

FIGS. 19G-19I show stages in a method of creating a cavity using a steerable injector with a plurality of cavity creation elements during a vertebroplasty procedure in accordance with the invention.

FIGS. 20A and 20F illustrate a steerable, curvable ablation catheter according to one embodiment of the invention.

FIGS. 20B-20D illustrate various cross-sections through the steerable, curvable ablation catheter of FIG. 20A, according to one embodiment of the invention.

FIG. 20E is a perspective view of one embodiment of an RF electrode.

FIG. 21 illustrates an alternative embodiment of a steerable, curvable ablation catheter with two radially extending electrodes.

FIG. 22 illustrates an alternative embodiment of a steerable, curvable ablation catheter with a retractable electrode feature.

FIGS. 23A-B schematically illustrate a method of creating a cavity using the steerable vertebroplasty ablation catheter, according to one embodiment of the invention.

FIG. 24 illustrates a steerable, curvable cryo ablation catheter according to one embodiment of the invention.

FIG. 25 illustrates a steerable, curvable cryo ablation catheter according to another embodiment of the invention.

FIGS. 26A-B schematically illustrate a method of creating a cavity using the steerable vertebroplasty cryo ablation catheter, according to one embodiment of the invention.

FIG. 27 illustrates a combined electrosurgical-cryosurgical steerable, curvable ablation catheter according to one embodiment of the invention.

### DETAILED DESCRIPTION OF THE PREFERRED EMBODIMENTS

The present invention provides improved delivery systems for delivery of a bone cement or bone cement composite for the treatment of vertebral compression fractures due to osteoporosis (OSP), osteo-trauma, and benign or malignant lesions such as metastatic cancers and myeloma, and associated access and deployment tools and procedures.

The primary materials in the preferred bone cement composite are methyl methacrylate and inorganic cancellous and/or cortical bone chips or particles. Suitable inorganic bone chips or particles are sold by Allosource, Osteotech and LifeNet (K053098); all have been cleared for marketing by FDA. The preferred bone cement also may contain the additives: barium sulfate for radio-opacity, benzoyl peroxide as an initiator, N,N-dimethyl-p-toluidine as a promoter and hydroquinone as a stabilizer. Other details of bone cements and systems are disclosed in U.S. patent application Ser. No. 11/626,336, filed Jan. 23, 2007, the disclosure of which is hereby incorporated in its entirety herein by reference.

One preferred bone cement implant procedure involves a two-step injection process with two different concentrations of the bone particle impregnated cement. To facilitate the implant procedure the bone cement materials are packaged in separate cartridges containing specific bone cement and inorganic bone particle concentrations for each step. Tables 1 and 2, infra, list one example of the respective contents and concentrations in Cartridges 1A and 1B for the first injection step, and Cartridges 2A and 2B for the second injection step.

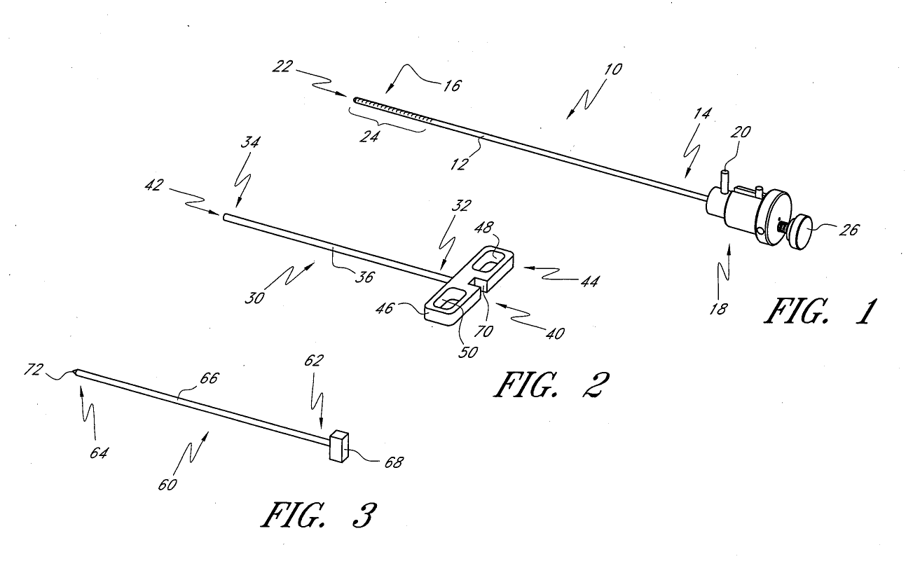

The bone cement delivery system generally includes at least three main components: 1) stylet; 2) introducer cannula; and 3) steerable injection needle. See FIGS. 1-3. Packaged with the system or packaged separately is a cement dispensing pump. The complete system also preferably includes at least one cement cartridge having at least two chambers therein, and a spiral mixing nozzle.

The stylet is used to perforate a hole into the pedicle of the vertebra to gain access to the interior of the vertebral body.

The introducer cannula is used for bone access and as a guide for the steerable injection needle. The introducer cannula is sized to allow physicians to perform vertebroplasty or kyphoplasty on vertebrae with small pedicles such as the thoracic vertebra T5 as well as larger vertebrae. In addition, this system is designed for uni-transpedicular access and/or bi-pedicular access.

Once bone access has been achieved, the steerable injection needle can be inserted through the introducer cannula into the vertebra. The entire interior vertebral body may be accessed using the steerable injection needle. The distal end of the needle can be manually shaped to any desired radius within the product specifications. The radius is adjusted by means of a knob on the proximal end of the device.

The hand-held cement dispensing pump may be attached to the steerable injection needle by a slip-ring luer fitting. The pre-filled 2-chambered cartridges (1A and 1B, and 2A and 2B) are loaded into the dispensing pump. As the handle of the dispensing pump is squeezed, each piston pushes the cartridge material into the spiral mixing tube. The materials are mixed in the spiral mixing nozzle prior to entering the steerable injection needle. The ratio of diameters of the cartridge chambers determines the mixing ratio for achieving the desired viscosity.

The bone cement implant procedures described herein use established vertebroplasty and kyphoplasty surgical procedures to stabilize the collapsed vertebra by injecting bone cement into cancellous bone.

The preferred procedure is designed for uni-transpedicular access and may be accomplished under either a local anesthetic or short-duration general anesthetic. Once the area of the spine is anesthetized, an incision is made and the stylet is used to perforate the vertebral pedicle and gain access to the interior of the vertebral body. The introducer cannula is then inserted and acts as a guide for the steerable injection needle.

Injection of the preferred bone cement involves a two-step procedure. The pre-filled Cartridges 1A and 1B are loaded into the dispensing pump. As the dispensing pump handle is squeezed, each piston pushes material into the spiral mixing tube. The diameter of each chamber may be utilized to determine the mixing ratio for achieving the desired viscosity.

The first step involves injecting a small quantity of PMMA with more than about 35%, e.g., 60% inorganic bone particles, onto the outer periphery of the cancellous bone matrix, i.e., next to the inner wall of the cortical bone of the vertebral body. The cement composite is designed to harden relatively quickly, forming a firm but still pliable shell. This shell is intended to prevent bone marrow/PMMA content from being ejected through any venules or micro-fractures in the vertebral body wall. The second step of the procedure involves a second injection of PMMA with an approximately 30% inorganic bone particles to stabilize the remainder of the weakened, compressed cancellous bone.

Alternatively, the steerable needle disclosed herein and discussed in greater detail below, can be used in conventional vertebroplasty procedures, using a single step bone cement injection.

Injection control for the first and second steps is provided by a 2 mm ID flexible injection needle, which is coupled to the hand operated bone cement injection pump. The 60% (>35%) and 30% ratio of inorganic bone particle to PMMA concentrations may be controlled by the pre-filled cartridge sets 1A and 1B, and 2A and 2B. At all times, the amount of the injectate is under the direct control of the surgeon or intervention radiologist and visualized by fluoroscopy. The introducer cannula is slowly withdrawn from the cancellous space as the second injection of bone cement begins to harden, thus preventing bone marrow/PMMA content from exiting the vertebral body. The procedure concludes with closure of the surgical incision with bone filler. In vitro and in vivo studies have shown that the 60% (>35%) bone-particle impregnated bone cement hardens in 2-3 minutes and 30% bone-particle impregnated bone cement hardens between 4 to 10 minutes.

Details of the system components will be discussed below.

There is provided in accordance with the present invention a steerable injection device that can be used to introduce any of a variety of materials or devices for diagnostic or therapeutic purposes. In one embodiment, the system is used to inject bone cement, e.g., PMMA or any of the bone cement compositions disclosed elsewhere herein. The injection system most preferably includes a tubular body with a steerable (i.e., deflectable) distal portion for introducing bone cement into various locations displaced laterally from the longitudinal axis of the device within a vertebral body during a vertebroplasty procedure.

Referring to FIG. 1, there is illustrated a side perspective view of a steerable injection needle 10 in accordance with one aspect of the present invention. The steerable injection needle 10 comprises an elongate tubular body 12 having a proximal end 14 and a distal end 16. The proximal end 14 is provided with a handle or manifold 18, adapted to remain outside of the patient and enable introduction and/or aspiration of bone cement or other media, and control of the distal end as will be described herein. In general, manifold 18 is provided with at least one injection port 20, which is in fluid communication with a central lumen (not illustrated) extending through tubular body 12 to at least one distal exit port 22.

The manifold 18 is additionally provided with a control 26 such as a rotatable knob, slider, or other moveable control, for controllably deflecting a deflection zone 24 on the distal end 16 of the tubular body 12. As is described elsewhere herein, the deflection zone 24 may be advanced from a relatively linear configuration as illustrated in FIG. 1 to a deflected configuration throughout an angular range of motion.

Referring to FIG. 2, there is illustrated an elongate tubular introducer 30, having a proximal end 32, a distal end 34 and an elongate tubular body 36 extending therebetween. A central lumen 38 (not shown) extends between a proximal access port 40 and a distal access port 42.

The central lumen 38 has an inside diameter which is adapted to slideably axially receive the steerable injection needle 10 therethrough. This enables placement of the distal end 34 adjacent a treatment site within the body, to establish an access pathway from outside of the body to the treatment site. As will be appreciated by those of skill in the art, the introducer 30 enables procedures deep within the body such as within the spine, through a minimally invasive and/or percutaneous access. The steerable injection needle 10 and/or other procedure tools may be introduced into port 40, through lumen 38 and out of port 42 to reach the treatment site.

The proximal end 32 of introducer 30 may be provided with a handle 44 for manipulation during the procedure. Handle 44 may be configured in any of a variety of ways, such as having a frame 46 with at least a first aperture 48 and a second aperture 50 to facilitate grasping by the clinician.

Referring to FIG. 3, there is illustrated a perspective view of stylet 60. Stylet 60 comprises a proximal end 62, a distal end 64 and an elongate body 66 extending therebetween. The proximal end 62 may be provided with a stop 68 such as a grasping block, manifold or other structure, to facilitate manipulation by the clinician. In the illustrated embodiment, the block 68 is configured to nest within a recess 70 on the proximal end of the introducer 30.

As will be appreciated by those of skill in the art, the stylet 60 has an outside diameter which is adapted to coaxially slide within the central lumen on introducer 30. When block 68 is nested within recess 70, a distal end 64 of stylet 60 is exposed beyond the distal end 34 of introducer 30. The distal end 64 of stylet 60 may be provided with a pointed tip 72, such as for anchoring into the surface of a bone.

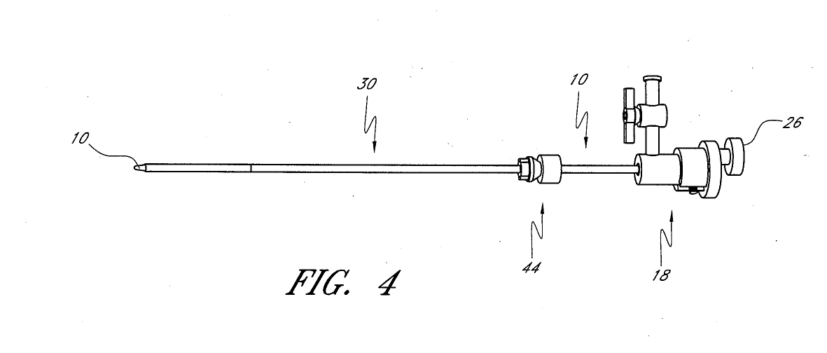

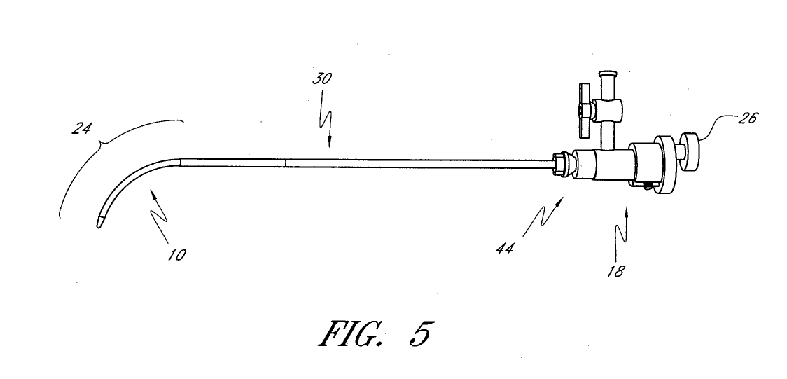

Referring to FIG. 4, there is illustrated a side elevational view of an assembly in accordance with the present invention in which a steerable injection needle 10 is coaxially positioned within an introducer 30. The introducer 30 is axially moveably carried on the steerable injection needle 10. In the illustration of FIG. 4, the introducer 30 is illustrated in a distal position such that it covers at least a portion of the deflection zone 24 on injection needle 10.

FIG. 5 illustrates an assembly as in FIG. 4, in which the introducer 30 has been proximally retracted along the injection needle 10 to fully expose the deflection zone 24 on injection needle 10. In addition, the control 26 has been manipulated to deflect the deflection zone 24 through an angle of approximately 90°. Additional details of the steerable needle will be discussed below.

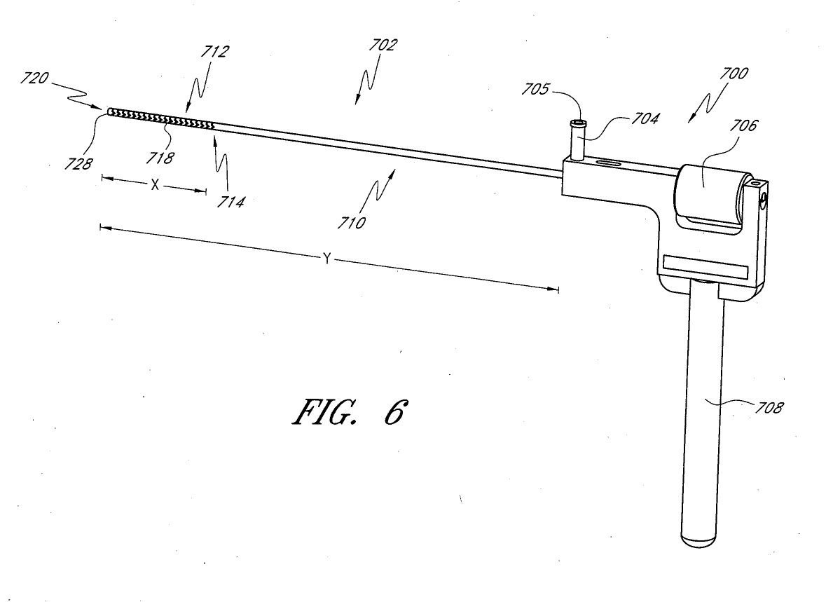

FIG. 6 illustrates a schematic perspective view of an alternate steerable vertebroplasty injector, according to one embodiment of the invention. The steerable injector 700 includes a body or shaft portion 702 that is preferably elongate and tubular, input port 704, adjustment control 706, and handle portion 708. The elongate shaft 702 preferably has a first proximal portion 710 and a second distal portion 712 which merge at a transition point 714. Shaft 702 may be made of stainless steel, such as 304 stainless steel, Nitinol, Elgiloy, or other appropriate material. Alternatively, the tubular body 702 may be extruded from any of a variety of polymers well known in the catheter arts, such as PEEK, PEBAX, nylon and various polyethylenes. Extruded tubular bodies 702 may be reinforced using metal or polymeric spiral wrapping or braided wall patterns, as is known in the art.

The shaft 702 defines at least one lumen therethrough that is preferably configured to carry a flowable bone cement prior to hardening. Proximal portion 710 of shaft 702 is preferably relatively rigid, having sufficient column strength to push through cancellous bone. Distal portion 712 of shaft 702 is preferably flexible and/or deflectable and reversibly actuatable between a relatively straight configuration and one or more deflected configurations or curved configurations as illustrated, for example, in FIG. 5, as will be described in greater detail below. The distal portion 712 of shaft 702 may include a plurality of transverse slots 718 that extend partially circumferentially around the distal portion 712 of the shaft 702 to provide a plurality of flexion joints to facilitate bending.

Input port 704 may be provided with a Luer lock connector although a wide variety of other connector configurations, e.g., hose barb or slip fit connectors can also be used. Lumen 705 of input port 704 is fluidly connected to central lumen 720 of shaft 702 such that material can flow from a source, through input port 704 into central lumen 720 of the shaft 702 and out the open distal end or out of a side opening on distal portion 712. Input port 704 is preferably at least about 20 gauge and may be at least about 18, 16, 14, or 12 gauge or larger in diameter.

Input port 704 advantageously allows for releasable connection of the steerable injection device 700 to a source of hardenable media, such as a bone cement mixing device described herein. In some embodiments, a plurality of input ports 704, such as 2, 3, 4, or more ports are present, for example, for irrigation, aspiration, introduction of medication, hardenable media precursors, hardenable media components, catalysts or as a port for other tools, such as a light source, cautery, cutting tool, visualization devices, or the like. A first and second input port may be provided, for simultaneous introduction of first and second bone cement components such as from a dual chamber syringe or other dispenser. A mixing chamber may be provided within the injection device 700, such as within the proximal handle, or within the tubular shaft 702

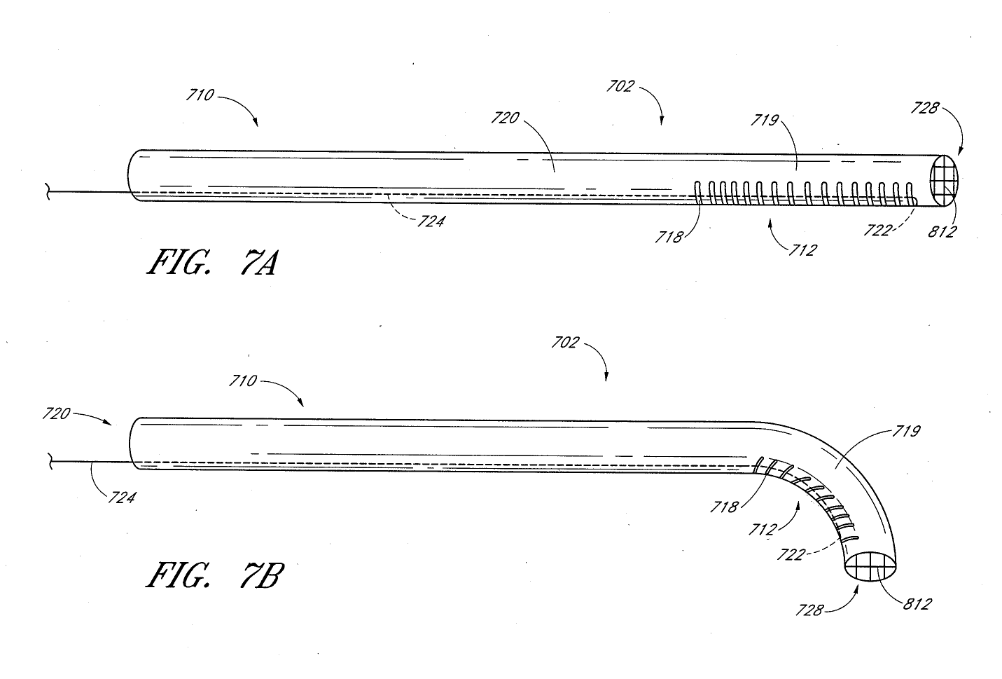

A variety of adjustment controls 706 may be used with the steerable injection system, for actuating the curvature of the distal portion 712 of the shaft 702. Preferably, the adjustment control 706 advantageously allows for one-handed operation by a physician. In one embodiment, the adjustment control 706 is a rotatable member, such as a thumb wheel or dial. The dial can be operably connected to a proximal end of an axially movable actuator such as pull wire 724. See FIG. 7A. When the dial is rotated in a first direction, a proximally directed tension force is exerted on the pull wire 724, actively changing the curvature of the distal portion 712 of the shaft 702 as desired. The degree of deflection can be observed fluoroscopically, and/or by printed or other indicium associated with the control 706. Alternative controls include rotatable knobs, slider switches, compression grips, triggers such as on a gun grip handle, or other depending upon the desired functionality.

In some embodiments, the adjustment control 706 allows for continuous adjustment of the curvature of the distal portion 712 of shaft 702 throughout a working range. In other embodiments, the adjustment control is configured for discontinuous (i.e., stepwise) adjustment, e.g., via a ratcheting mechanism, preset slots, deflecting stops, a rack and pinion system with stops, ratcheting band (adjustable zip-tie), adjustable cam, or a rotating dial of spring loaded stops. In still other embodiments, the adjustment control 706 may include an automated mechanism, such as a motor or hydraulic system to facilitate adjustment.

The adjustment control may be configured to allow deflection of the distal portion 712 through a range of angular deviations from 0 degrees (i.e., linear) to at least about 15°, and often at least about 25°, 35°, 60°, 90°, 120°, 150°, or more degrees from linear.

In some embodiments, the length X of the flexible distal portion 712 of shaft 702 is at least about 10%, in some embodiments at least about 15%, 25%, 35%, 45%, or more of the length Y of the entire shaft 702 for optimal delivery of bone cement into a vertebral body. One of ordinary skill in the art will recognize that the ratio of lengths X:Y can vary depending on desired clinical application. In some embodiments, the maximum working length of needle 702 is no more than about 15″, 10″, 8″, 7″, 6″, or less depending upon the target and access pathway. In one embodiment, when the working length of needle 702 is no more than about 8″, the adjustable distal portion 712 of shaft has a length of at least about 1″ and preferably at least about 1.5″ or 2″.

FIGS. 7A-B are schematic perspective views of a distal portion of shaft 702 of a steerable vertebroplasty injector, according to one embodiment of the invention. Shown is the preferably rigid proximal portion 710 and deflectable distal portion 712. The distal portion 712 of shaft 702 includes a plurality of transverse slots 718 that extend partially circumferentially around the distal portion 712 of the shaft 702, leaving a relatively axially non-compressible spine 719 in the form of the unslotted portion of the tubular wall.

In some embodiments, the slots 718 can be machined or laser cut out of the tube stock that becomes shaft 702, and each slot may have a linear, chevron or other shape. In other embodiments, the distal portion 712 of shaft 702 may be created from an elongate coil rather than a continuous tube.

Slots 718 provide small compression hinge joints to assist in the reversible deflection of distal portion 712 of shaft 702 between a relatively straightened configuration and one or more curved configurations. One of ordinary skill in the art will appreciate that adjusting the size, shape, and/or spacing of the slots 718 can impart various constraints on the radius of curvature and/or limits of deflection for a selected portion of the distal portion 712 of shaft 702. For example, the distal portion 712 of shaft 702 may be configured to assume a second, fully deflected shape with a relatively constant radius of curvature throughout its length. In other embodiments, the distal portion 712 may assume a progressive curve shape with a variable radius of curvature which may, for example, have a decreasing radius distally. In some embodiments, the distal portion may be laterally displaced through an arc having a radius of at least about 0.5″, 0.75″, 1.0″, 1.25″, or 1.5″ minimum radius (fully deflected) to ∞ (straight) to optimize delivery of bone cement within a vertebral body. Wall patterns and deflection systems for bendable slotted tubes are disclosed, for example, in U.S. Patent Publication No. 2005/0060030 A1 to Lashinski et al., the disclosure of which is incorporated in its entirety by reference herein.

Still referring to FIGS. 7A-B, a pull wire 724 resides within the lumen 720 of shaft 702. The distal end 722 of the pull wire 724 is preferably operably attached, such as by adhesive, welding, soldering, crimping or the like, to an inner side wall of the distal portion 712 of the shaft 702. Preferably, the attachment point will be approximately 180° offset from the center of the axially extending spine 719. Proximal portion of pull wire 724 is preferably operably attached to adjustment control 706. The adjustment control 706 may be configured to provide an axial pulling force in the proximal direction toward the proximal end of pull wire 724. This in turn exerts a proximal traction on the distal portion 712 of shaft 702 operably attached to distal end 722 of pull wire 724. The slotted side of the tubular body shortens under compression, while the spine side 719 retains its axial length causing the distal portion 712 of shaft 702 to assume a relatively curved or deflected configuration. In some embodiments, a plurality of pull wires, such as two, three, four, or more pull wires 724 may be present within the lumen 720 with distal points of attachment spaced axially apart to allow the distal portion 712 of shaft 702 to move through compound bending curves depending on the desired bending characteristic. Distal axial advance of the actuator will cause a deflection in an opposite direction, by increasing the width of the slots 718.

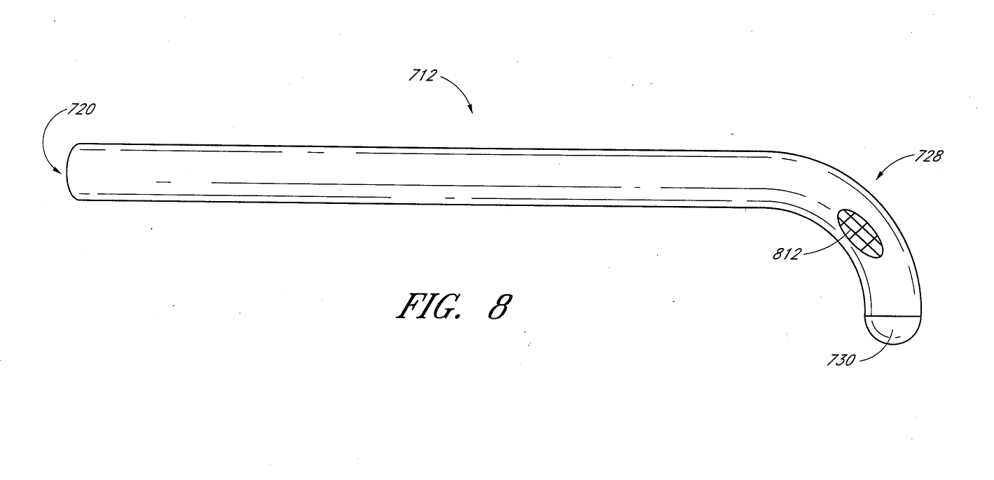

A distal opening 728 is provided on shaft 702 in communication with central lumen 720 to permit expression of material, such as bone cement, from the injector 700. Some embodiments may include a filter such as mesh 812. Mesh structure 812 can advantageously control cement output by controlling bubbles and/or preventing undesired large or unwieldy aggregations of bone cement from being released at one location and thus promote a more even distribution of bone cement within the vertebral body. The mesh 812 may be created by a laser-cut cris-crossing pattern within distal end as shown, or can alternatively be separately formed and adhered, welded, or soldered on to the distal opening 728. Referring to FIG. 8, the distal shaft portion 712 may also include an end cap 730 or other structure for occluding central lumen 720, and a distal opening 728 on the sidewall of shaft 702.

In some embodiments, the distal shaft 712 can generate a lateral force of at least about 0.125 pounds, 0.25 pounds, 0.5 pounds, 1 pound, 1.5 pounds, 2 pounds, 3 pounds, 4 pounds, 5 pounds, 6 pounds, 7 pounds, 8 pounds, 9 pounds, 10 pounds, or more by activating control 706. This can be advantageous to ensure that the distal portion 712 is sufficiently navigable laterally through cancellous bone to distribute cement to the desired locations. In some embodiments, the distal shaft 712 can generate a lateral force of at least about 0.125 pounds but no more than about 10 pounds; at least about 0.25 pounds but no more than about 7 pounds; or at least about 0.5 pounds but no more than about 5 pounds.

In some embodiments, the distal portion 712 of shaft 702 (or end cap 730) has visible indicia, such as, for example, a marker visible via one or more imaging techniques such as fluoroscopy, ultrasound, CT, or MRI.

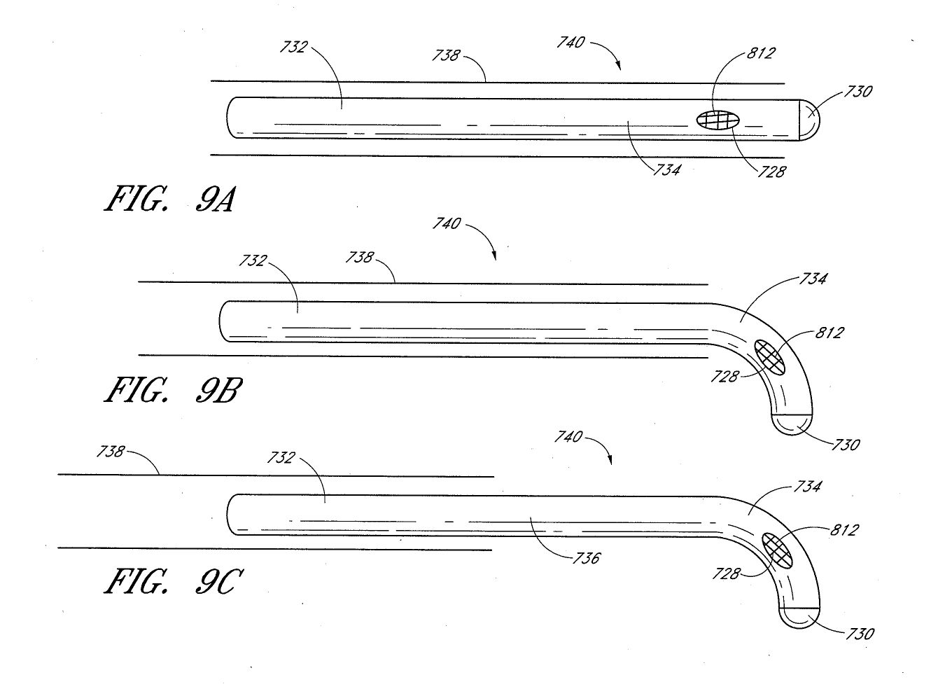

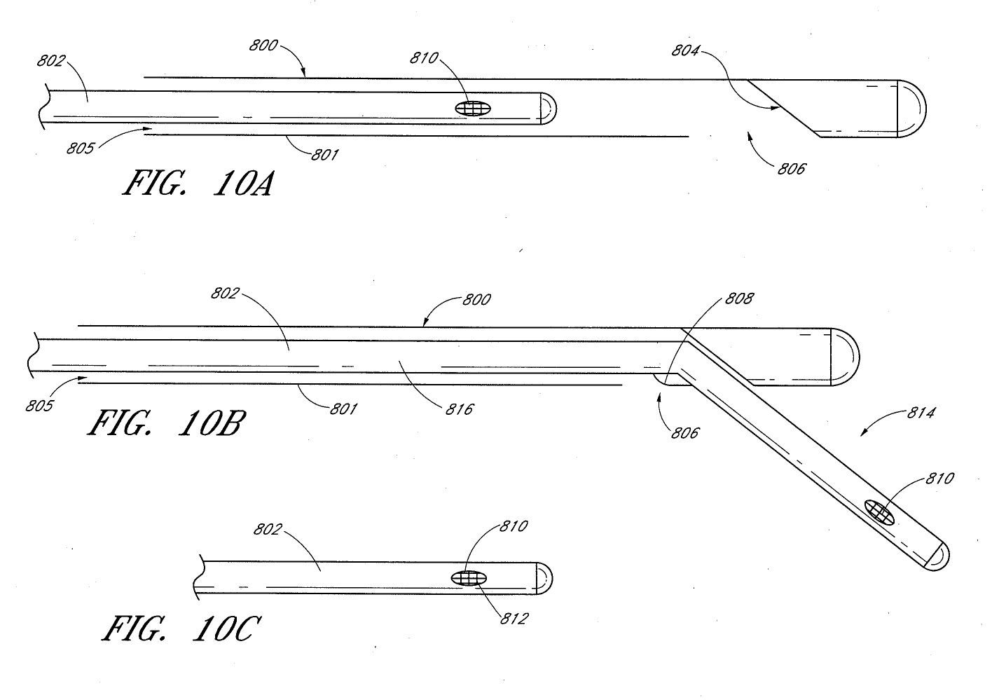

FIGS. 9A-C illustrate in schematic cross-section another embodiment of a distal portion 734 of a steerable injection device 740. The tubular shaft 736 can include a distal portion 734 made of or containing, for example, a shape memory material that is biased into an arc when in an unconstrained configuration. Some materials that can be used for the distal curved portion 734 include Nitinol, Elgiloy, stainless steel, or a shape memory polymer. A proximal portion 732 of the shaft 736 is preferably relatively straight as shown. Also shown is end cap 730, distal lateral opening 728 and mesh 812.

The distal curved portion 734 may be configured to be axially movably received within an outer tubular sheath 738. The sheath 738 is preferably configured to have sufficient rigidity and radial strength to maintain the curved distal portion 734 of shaft 732 in a relatively straightened configuration while the outer tubular sheath 738 coaxially covers the curved distal portion 734. Sheath 738 can be made of, for example, a metal such as stainless steel or various polymers known in the catheter arts. Axial proximal withdrawal of the sheath 738 with respect to tubular shaft 736 will expose an unconstrained portion of the shape memory distal end 734 which will revert to its unstressed arcuate configuration. Retraction of the sheath 738 may be accomplished by manual retraction by an operator at the proximal end, retraction of a pull wire attached to a distal portion of the sheath 738, or other ways as known in the art. The straightening function of the outer sheath 738 may alternatively be accomplished using an internal stiffening wire, which is axially movably positionable within a lumen extending through the tubular shaft 736. The length, specific curvature, and other details of the distal end may be as described elsewhere herein.

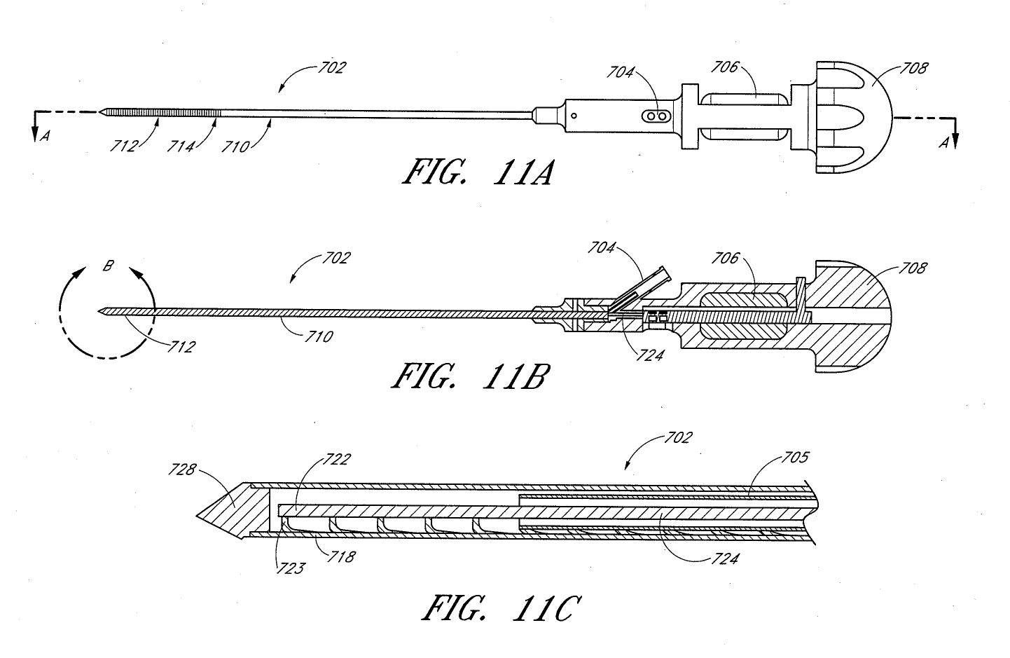

In another embodiment, as shown in FIGS. 10A-C, tubular shaft 802 of a steerable vertebroplasty injector may be generally substantially straight throughout its length in its unstressed state, or have a laterally biased distal end. A distally facing or side facing opening 810 is provided for the release of a material, such as bone cement. In this embodiment, introducer 800 includes an elongate tubular body 801 with a lumen 805 therethrough configured to receive the tubular shaft (also referred to as a needle) 802. Introducer 800 can be made of any appropriate material, such as, stainless steel and others disclosed elsewhere herein. Needle 802 may be made of a shapememory material, such as Nitinol, with superelastic properties, and has an outside diameter within the range of between about 1 to about 3 mm, about 1.5-2.5 mm, or about 2.1 mm in some embodiments.

Introducer 800 includes a needle-redirecting element 804 such as an inclined surface near its distal end. Needle-redirecting element 804 can be, for example, a laser-cut tang or a plug having a proximal surface configured such that when needle 802 is advanced distally into introducer 800 and comes in contact with the needle-redirecting element 804, a distal portion 814 of needle 802 is redirected out an exit port 806 of introducer 800 at an angle 808, while proximal portion 816 of needle 802 remains in a relatively straightened configuration, as shown in FIG. 10B. Bone cement can then be ejected from distal opening 810 on the end or side of needle 802 within bone 1000. Distal opening 810 may be present at the distal tip of the needle 802 (coaxial with the long axis of the needle 802) or alternatively located on a distal radial wall of needle 802 as shown in FIG. 10C. In some embodiments, the angle 808 is at least about 15 degrees and may be at least about 30, 45, 60, 90, 105 degrees or more with respect to the long axis of the introducer 800.

The illustrated embodiment of FIGS. 10A-C and other embodiments disclosed herein are steerable through multiple degrees of freedom to distribute bone cement to any area within a vertebral body. For example, the introducer 800 and needle 802 can both rotate about their longitudinal axes with respect to each other, and needle 802 can move coaxially with respect to the introducer 800, allowing an operator to actuate the injection system three dimensionally. The distal portion 814 of needle 802 can be deflected to a position that is angularly displaced from the long axis of proximal portion 816 of needle without requiring a discrete curved distal needle portion as shown in other embodiments herein.

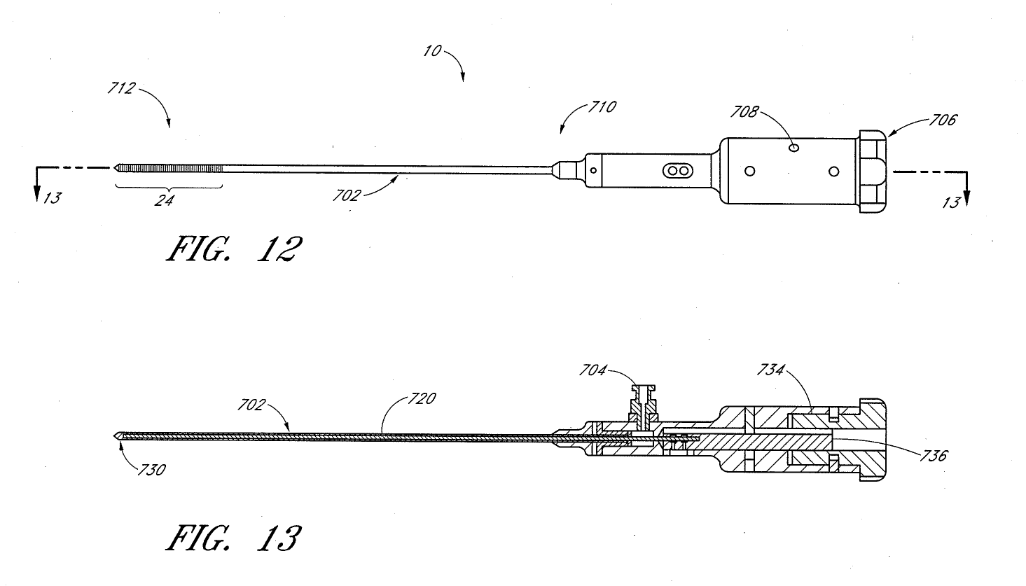

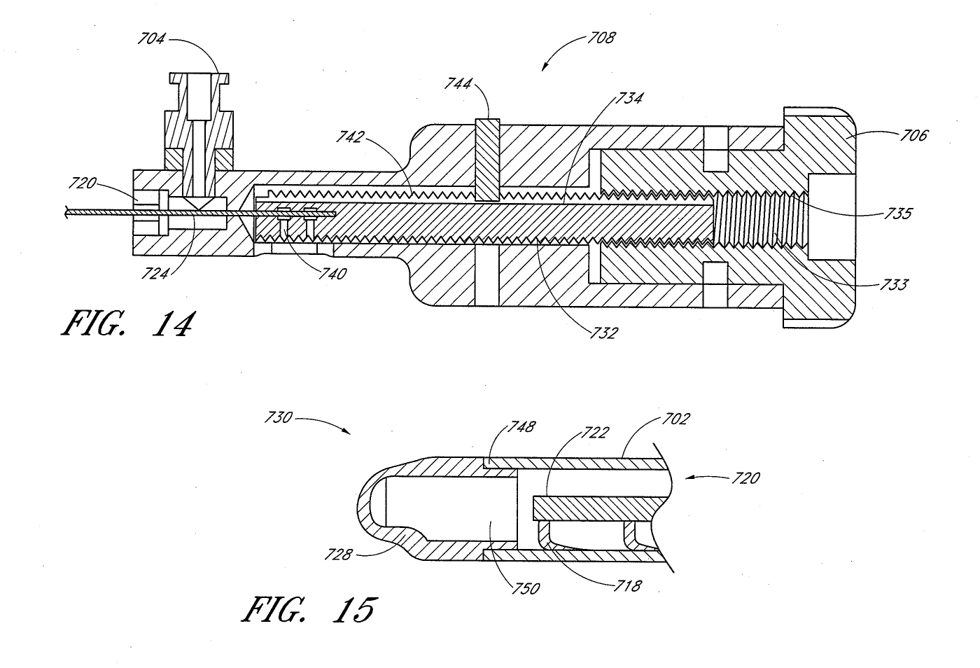

FIGS. 11A-C illustrate another embodiment of a steerable vertebroplasty injector. FIG. 11A schematically shows handle portion 708, adjustment control 706, and elongate needle shaft 702, including proximal portion 710, distal portion 712, and transition point 714. FIG. 11B is a vertical cross-section through line A-A of FIG. 11A, and shows adjustment control 706 operably connected to pull wire 724 such as through a threaded engagement. Also shown is input port 704, and proximal portion 710 and distal portion 712 of needle shaft 702. FIG. 11C illustrates a cross-sectional view of distal portion 712 of shaft 702. The distal end 722 of pull wire 724 is attached at an attachment point 723 to the distal portion 712 of shaft 702. Proximal retraction on pullwire 724 will collapse transverse slots 718 and deflect the injector as has been discussed. Also shown is an inner tubular sleeve 709, which can be advantageous to facilitate negotiation of objects or media such as bone cement, through the central lumen of the needle shaft 702.

The interior sleeve 709 is preferably in the form of a continuous, tubular flexible material, such as nylon or polyethylene. In an embodiment in which the needle 702 has an outside diameter of 0.095 inches (0.093 inch coil with a 0.001 inch thick outer sleeve) and an inside diameter of 0.077 inches, the interior tubular sleeve 709 may have an exterior diameter in the area of about 0.074 inches and an interior diameter in the area of about 0.069 inches. The use of this thin walled tube 705 on the inside of the needle shaft 702 is particularly useful for guiding a fiber through the needle shaft 702. The interior tube 705 described above is additionally preferably fluid-tight, and can be used to either protect the implements transmitted therethrough from moisture, or can be used to transmit bone cement through the steerable needle.

In some embodiments, an outer tubular coating or sleeve (not shown) is provided for surrounding the steerable needle shaft at least partially throughout the distal end of the needle. The outer tubular sleeve may be provided in accordance with techniques known in the art and, in one embodiment, is a thin wall polyester (e.g., ABS) heat shrink tubing such as that available from Advanced Polymers, Inc. in Salem, N.H. Such heat shrink tubings have a wall thickness of as little as about 0.0002 inches and tube diameter as little as about 0.010 inches. The outer tubular sleeve enhances the structural integrity of the needle, and also provides a fluid seal and improved lubricity at the distal end over embodiments with distal joints 718. Furthermore, the outer tubular sleeve tends to prevent the device from collapsing under a proximal force on a pull wire. The sleeve also improves pushability of the tubular members, and improves torque transmission.

In other embodiments, instead of a slotted tube, the needle shaft of a vertebroplasty injection system may include a metal or polymeric coil. Steerable helical coil-type devices are described, for example, in U.S. Pat. No. 5,378,234 or 5,480,382 to Hammerslag et al., which are both incorporated by reference herein in their entirety.

An interior tubular sleeve (not illustrated) may be provided to facilitate flow of media through the central lumen as described elsewhere in the application. In some embodiments, a heat-shrink outer tubular sleeve as described elsewhere in the application is also provided to enhance the structural integrity of the sheath, provide a fluid seal across the chevrons or slots, as well as improve lubricity.

The steerable injection needle (also referred to as the injection shaft) may have an outside diameter of between about 8 to 24 gauge, more preferably between about 10 to 18 gauge, e.g., 12 gauge, 13 gauge (0.095″ or 2.41 mm), 14 gauge, 15 gauge, or 16 gauge. In some embodiments, the inside diameter (luminal diameter) of the injection needle is between about 9 to 26 gauge, more preferably between about 11 to 19 gauge, e.g., 13 gauge, 14 gauge, 15 gauge, 16 gauge, or 17 gauge. In some embodiments, the inside diameter of the injection needle is no more than about 4 gauge, 3 gauge, 2 gauge, or 1 gauge smaller than the outside diameter of the injection needle.

The inside luminal diameter of all of the embodiments disclosed herein is preferably optimized to allow a minimal exterior delivery profile while maximizing the amount of bone cement that can be carried by the needle. In one embodiment, the outside diameter of the injection needle is 13 gauge (0.095″ or 2.41 mm) with a 0.077″ (1.96 mm) lumen. In some embodiments, the percentage of the inside diameter with respect to the outside diameter of the injection needle is at least about 60%, 65%, 70%, 75%, 80%, 85%, or more.

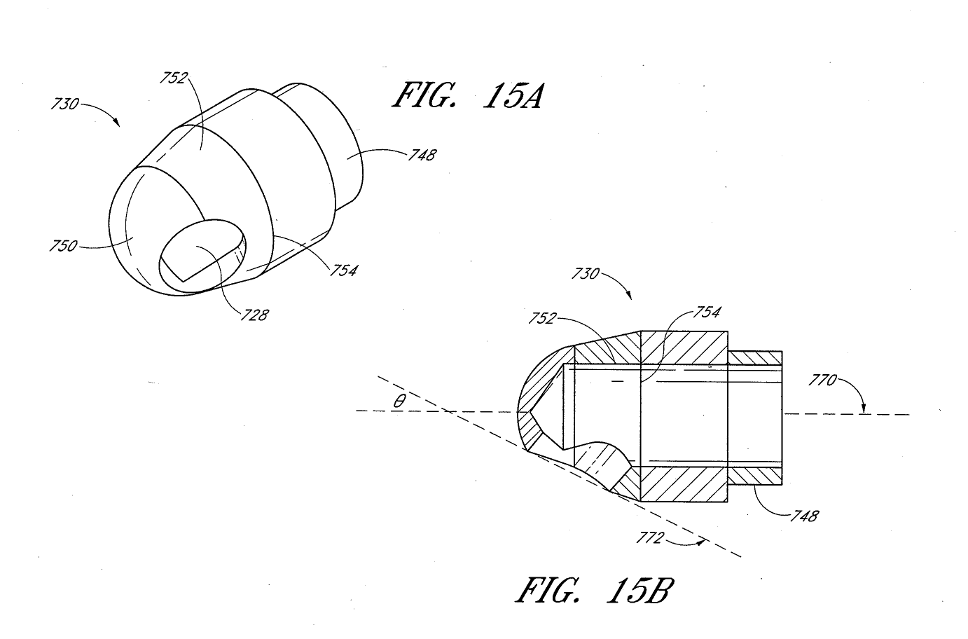

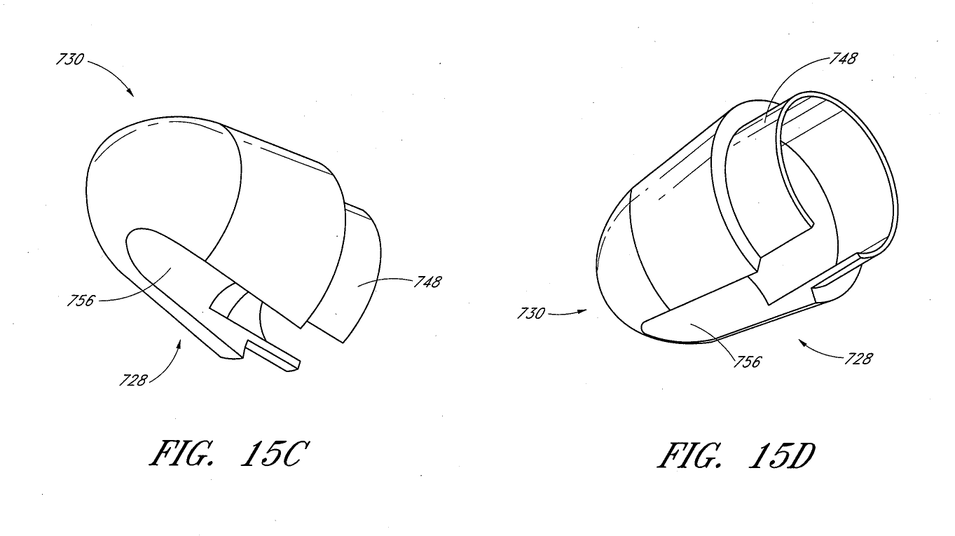

Referring to FIGS. 12 and 13, there is illustrated a modification of the steerable injection needle 10, in accordance with the present invention. The injection needle 10 comprises an elongate tubular shaft 702, extending between a proximal portion 710 and a distal portion 712. The proximal portion 710 is carried by a proximal handle 708, which includes a deflection control 706 such as a rotatable knob or wheel. Rotation of the control 706 causes a lateral deflection or curvature of the distal steering region 24 as has been discussed.

Input port 704 is in fluid communication with a distal opening 728 on a distal tip 730, by way of an elongate central lumen 720. Input port 704 may be provided with any of a variety of releasable connectors, such as a luer or other threaded or mechanically interlocking connector known in the art. Bone cement or other media advanced through lumen 720 under pressure may be prevented from escaping through the plurality of slots 718 in the steering region 24 by the provision of a thin flexible tubular membrane carried either by the outside of tubular shaft 702, or on the interior surface defining central lumen 720.

Referring to FIG. 14, the handle 708 is provided with an axially oriented central bore 732 having a first, female thread 733 thereon. A slider 734 having a second complementary male thread 735, is threadably engaged with the central bore 732. Rotation of the knob 706 relatively to the slider 734 thus causes the slider 734 to distally advance or proximally retract in an axial direction with respect to the handle 708. The slider 734 is mechanically linked to the pull wire 724, such as by the use of one or more set screws or other fastener 740.

Slider 734 is provided with at least one axially extending keyway or spline 742 for slideably engaging a slide dowel pin 744 linked to the handle 708. This allows rotation of the rotatable control 706, yet prevents rotation of the slider 734 while permitting axial reciprocal movement of the slider 734 as will be apparent to those of skill in the art. One or more actuating knob dowel pins 746 permits rotation of the rotatable control 706 with respect to the handle 708 but prevents axial movement of the rotatable control 706 with respect to the handle 708.

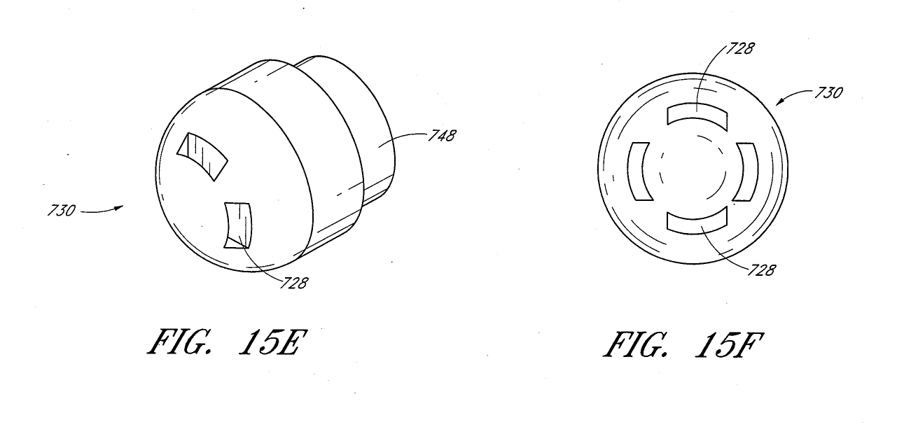

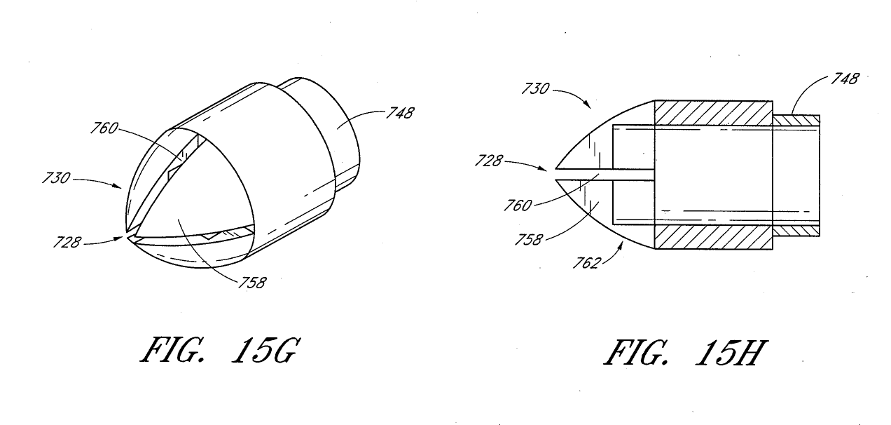

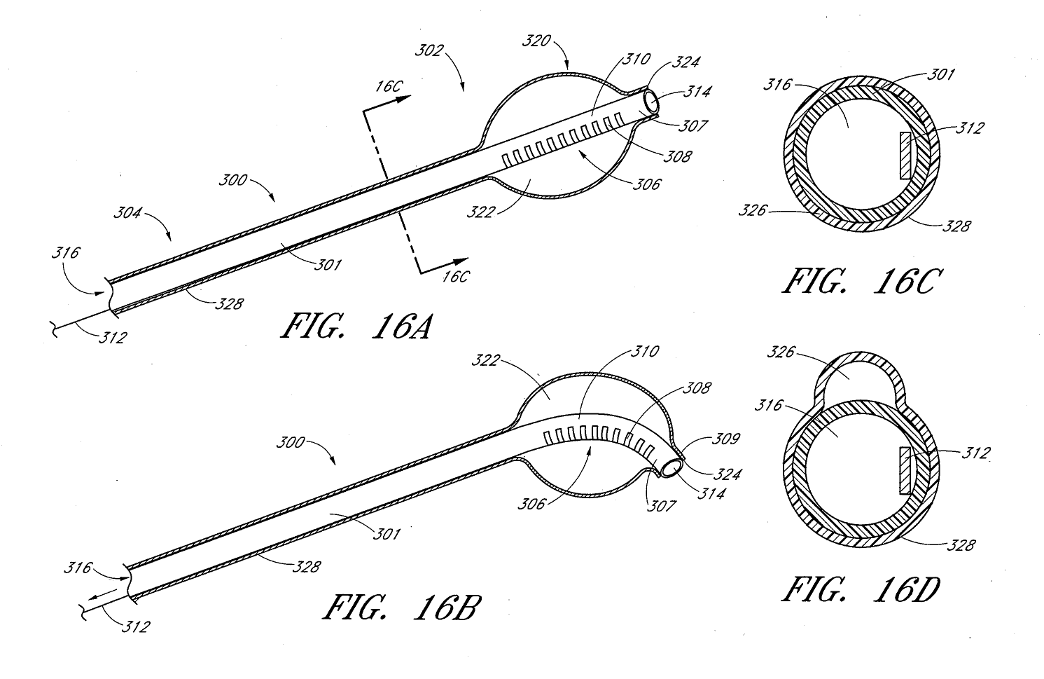

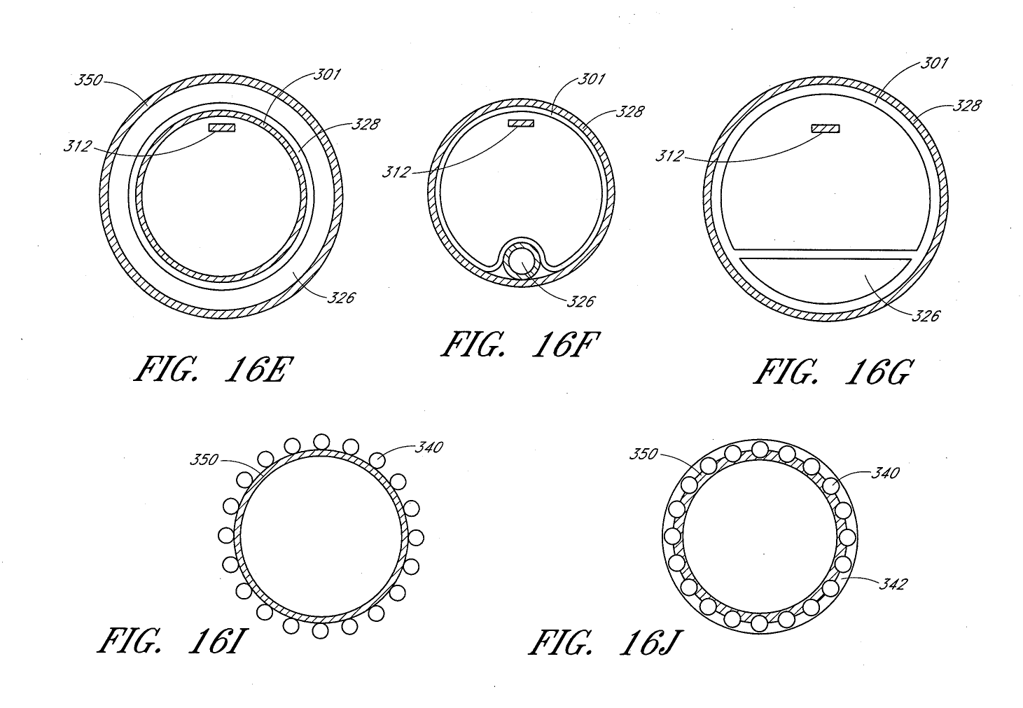

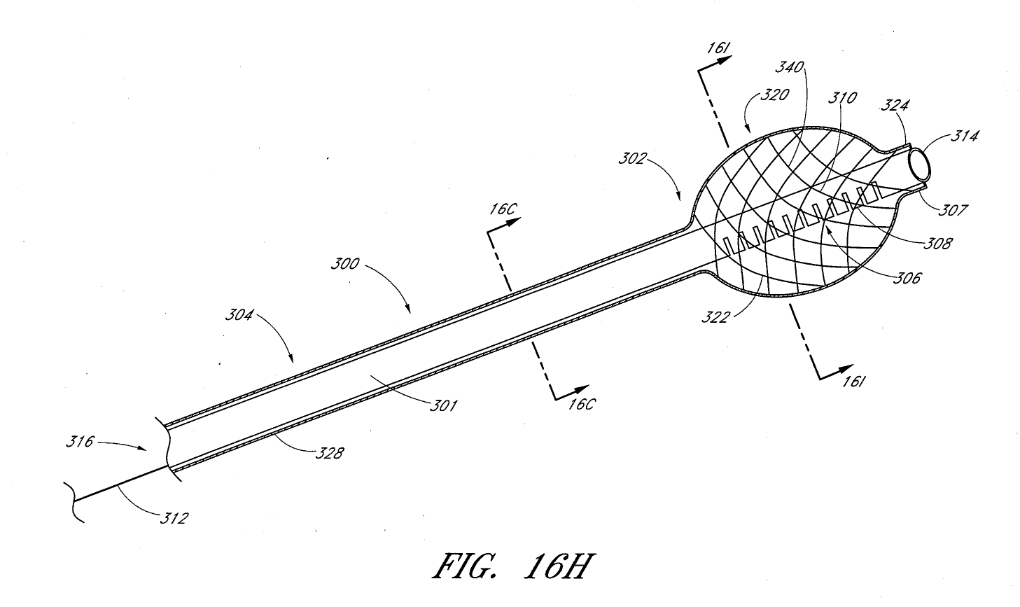

Referring to FIG. 15, the distal end of the shaft 702 may be provided with any of a variety of distal opening 728 orientations or distal tip 730 designs, depending upon the desired functionality. In the illustrated embodiment, the distal tip 730 is provided with an annular flange 748 which may be slip fit into the distal end of the tubular body 702, to facilitate attachment. The attachment of the distal tip 730 may be further secured by welding, crimping, adhesives, or other bonding technique.

In general, the distal tip 730 includes a proximal opening 750 for receiving media from the central lumen 720, and advancing media through distal opening 728. Distal opening 728 may be provided on a distally facing surface, on a laterally facing surface, or on an inclined surface of the distal tip 730.

Referring to FIGS. 15A and 15B, there is illustrated a distal tip 30 having a single inclined opening 728 thereon. In any of the designs disclosed herein, one or two or three or four or more distal ports 728 may be provided, depending upon the desired clinical performance. In the illustrated embodiment, the distal tip includes a rounded distal end 750 which transitions either smoothly or through an angular interface with an inclined portion 752. The distal opening 728 is positioned distally of a transition 754 at the proximal limit of the inclined surface 752. This configuration enables the distal opening 728 to have a distal axially facing component, as compared to an embodiment having a side wall opening. See, for example, FIG. 8.

Referring to FIG. 15B, the tip 730 can be considered to have a central longitudinal axis 770. The aperture 728 may be considered as residing on an aperture plane 772, which intersects the distal most limit and the proximal most limit of the aperture 728. Aperture plane 772 intersects the longitudinal axis at an angle θ. In an embodiment having a side wall aperture, the aperture plane 772 and longitudinal axis 770 would be parallel. In an embodiment having a completely distally facing aperture, the aperture plane 772 would intersect the longitudinal axis 770 at an angle of 90°.

In the illustrated embodiment, the inclined aperture 728 is defined by an aperture plane 772 intersecting the longitudinal axis 770 at an angle θ which is at least about 5°, often at least about 15°, and in many embodiments, at least about 25° or more. Intersection angles within the range of from about 15° to about 45° may often be used, depending upon the desired clinical performance.

Referring to FIGS. 15C and 15D, an alternate distal tip 730 is illustrated. In this configuration, the distal opening 728 is in the form of a sculpted recess 756 extending axially in alignment with at least a portion of the central lumen 720. Sculpted recess 756 may be formed in any of a variety of ways, such as by molding, or by drilling an axial bore in an axial direction with respect to the tip 730. The sculpted recess 756 cooperates with the tubular body 702, as mounted, to provide a distal opening 728 having an inclined aspect as well as an axially distally facing aspect with respect to the longitudinal axis of the steerable needle.

Referring to FIGS. 15E and 15F, there is illustrated a distal tip 730 having a plurality of distally facing apertures 728. In the illustrated embodiment, four distal apertures are provided. The distal apertures 728 may be provided on the rounded distal end 750, or on an inclined surface 752 as has been discussed.

Referring to FIGS. 15G and 15H, there is illustrated an alternative distal tip 730. In this configuration, an opening 728 is oriented in a distally facing direction with respect to the longitudinal axis of the needle. The distal opening of the central lumen is covered by at least one, preferably two, and, as illustrated, four leaflets 758 to provide a collet like configuration. Each of the adjacent leaflets 758 is separated by a slot 760 and is provided with a living hinge or other flexible zone 762.

In use, the distal tip 730 may be distally advanced through soft tissue, cortical or cancellous bone, with the distal opening 728 being maintained in a closed orientation. Following appropriate positioning of the distal tip 30, the introduction of bone cement or other media under pressure through the central lumen 720 forces the distal opening 728 open by radially outwardly inclining each leaflet 758 about its flection point 762. This configuration enables introduction of the needle without “coring” or occluding with bone or other tissue, while still permitting injection of bone cement or other media in a distal direction.

Any of the forgoing or other tip configurations may be separately formed and secured to the distal end of the tubular body 702, or may be machined, molded or otherwise formed integrally with the tube 702.

Alternatively, a distal opening aperture may be occluded by a blunt plug or cap, which prevents coring during distal advance of the device. Once positioned as desired, the distal cap may be pushed off of the distal end of the injector such as under the pressure of injected bone cement. The deployable cap may take any of a variety of forms depending upon the injector design. For example, it may be configured as illustrated in FIG. 15A, only without the aperture 728. The flange 748 is slip fit within the distal end of the injector body, and retained only by friction, or by a mild bond which is sufficient to retain the cap 730 during manipulation of the injector, but insufficient to resist the force of injected bone cement. The deployable cap 730 may be made from any of a variety of materials, such as stainless steel, Nitinol, or other implantable metals; any of a wide variety of implantable polymers such as PEEK, nylon, PTFE; or of bone cement such as PMMA. Alternatively, any of a variety of bioabsorbable polymers may be utilized to form the deployable cap 730, including blends and polymers in the PLA-PGLA absorbable polymer families.

As a further alternative, coring during insertion of an injector having a distal opening may be prevented by positioning a removable obturator in the distal opening. The obturator comprises an elongate body, extending from a proximal end throughout the length of the injector to a blunt distal tip. The obturator is advanced axially in a distal direction through the central lumen, until the distal tip of the obturator extends slightly distally of the distal opening in the injector. This provides a blunt atraumatic tip for distal advance of the injector through tissue. Following positioning of the injector, the obturator may be proximally withdrawn from the central lumen, and discarded. The obturator may be provided with any of a variety of structures for securing the obturator within the central lumen during the insertion step, such as a proximal cap for threadably engaging a complementary luer connector on the proximal opening of the central lumen.

In accordance with another aspect of the present invention, there is provided a combination device in which a steerable injector is additionally provided with a cavity formation element. Thus, the single device may be advanced into a treatment site within a bone, expanded to form a cavity, and used to infuse bone cement or other media into the cavity. Either or both of the expansion step and the infusion step may be accomplished following or with deflection of the distal portion of the injector.

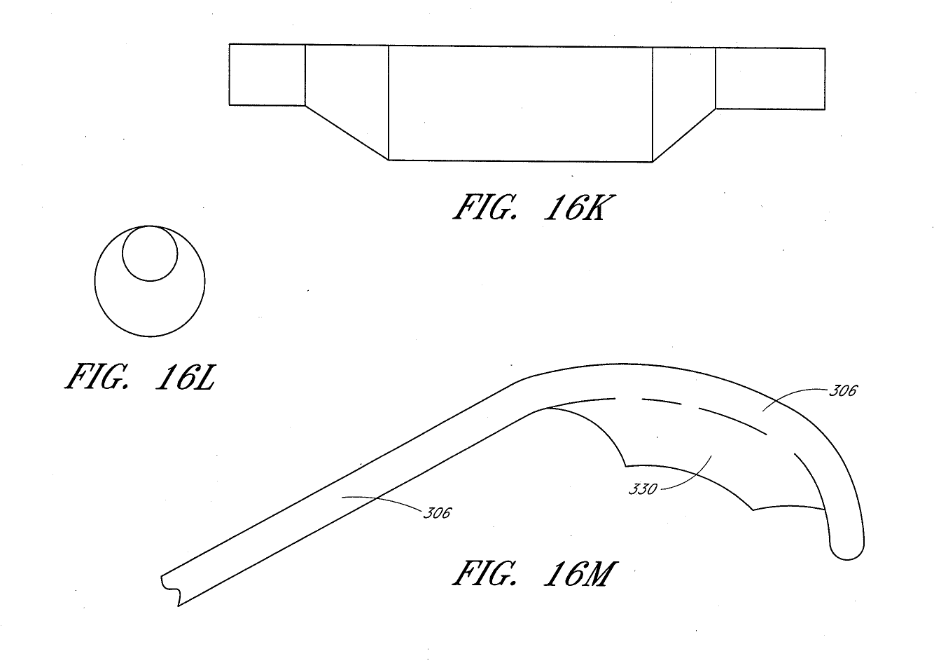

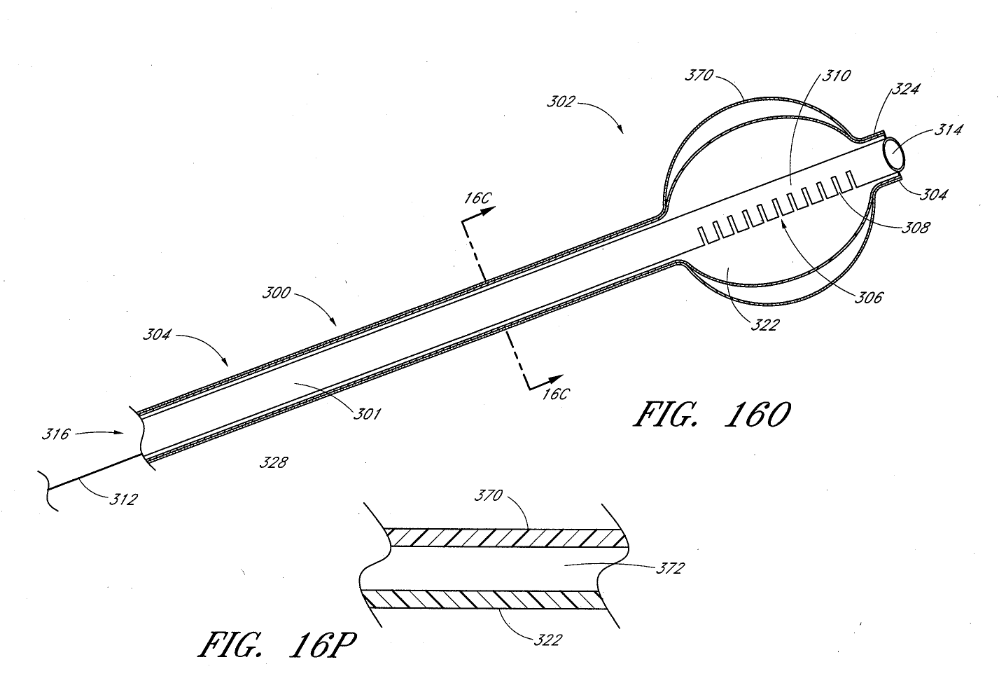

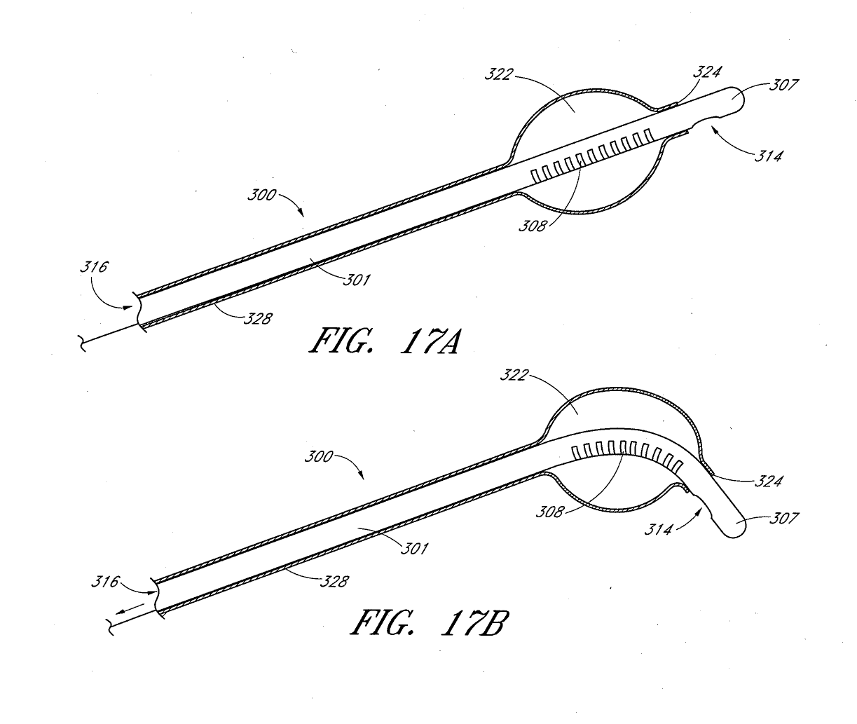

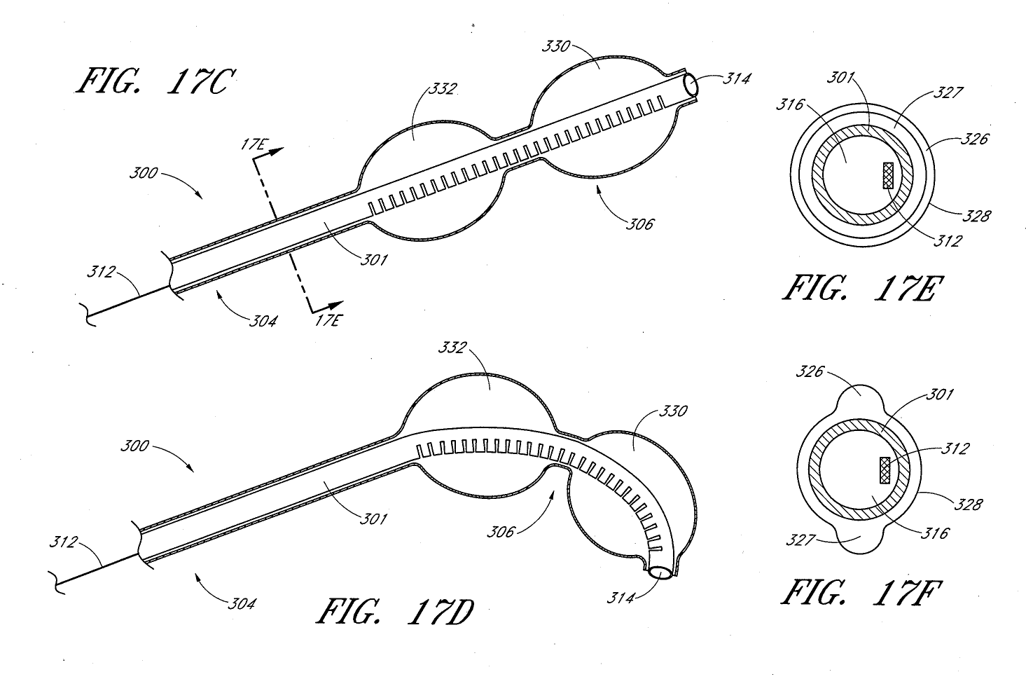

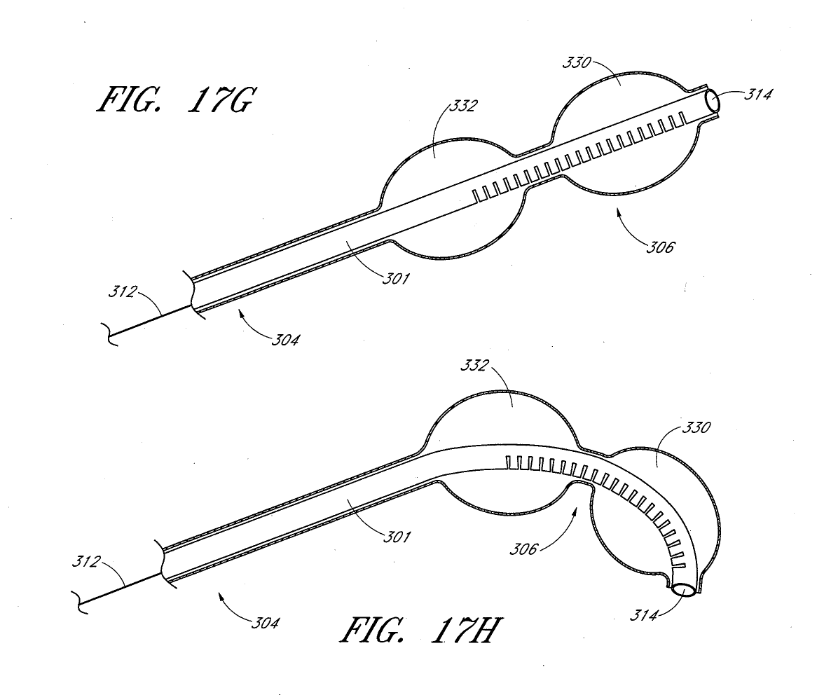

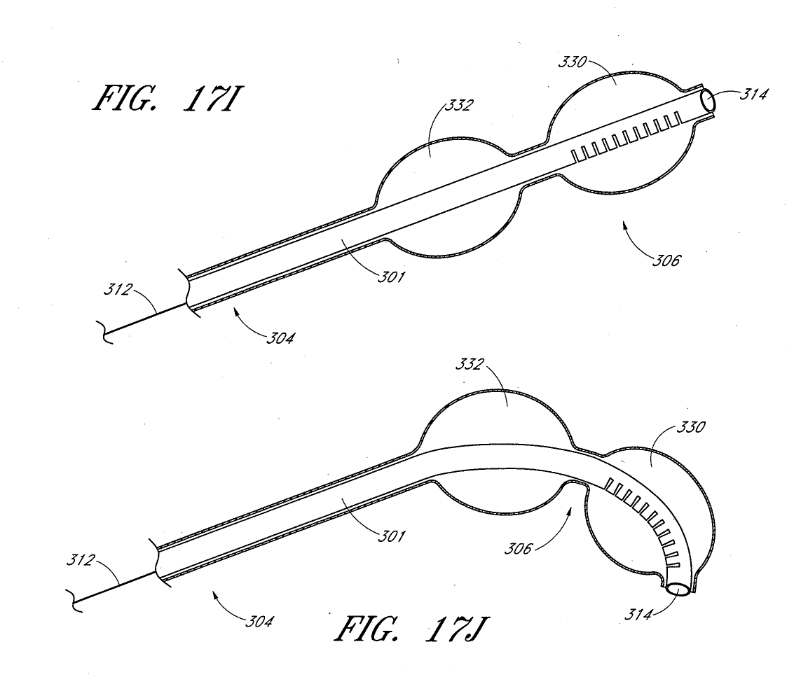

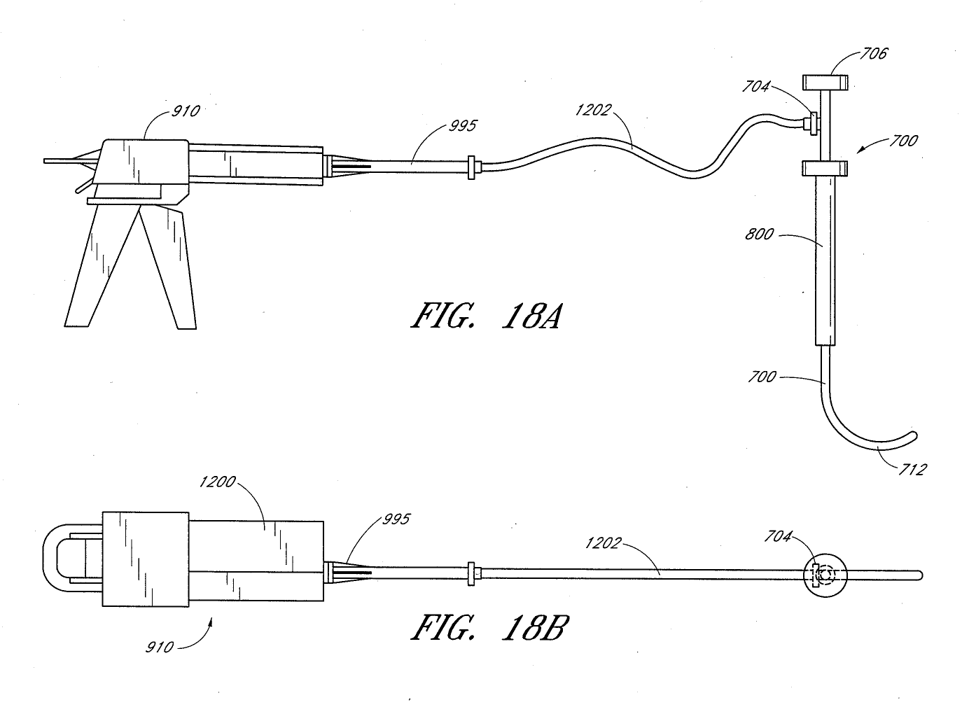

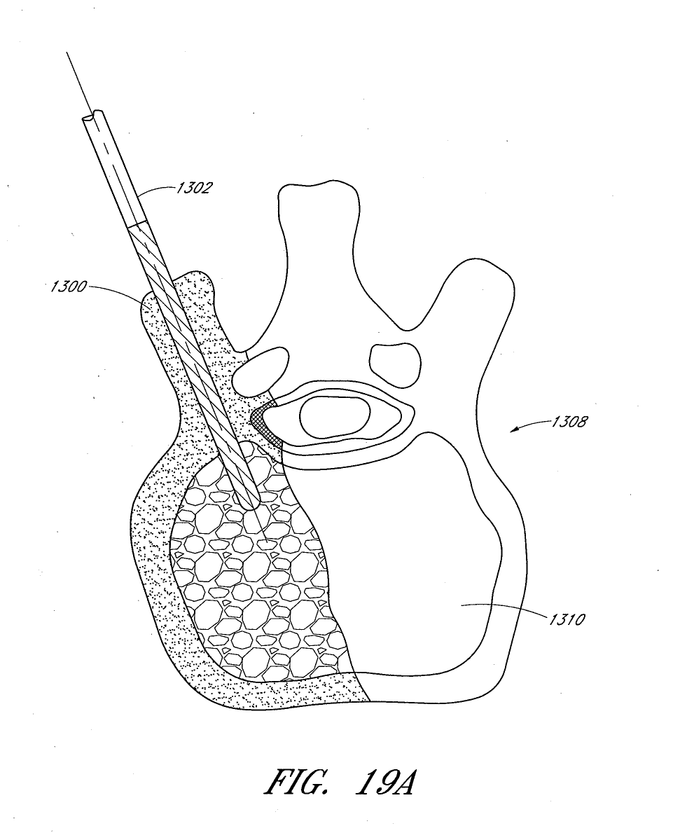

Referring to FIGS. 16A and 16B, the distal portion 302 of a steerable injector 300 having a cavity formation element 320 thereon is schematically illustrated. The steerable injector 300 includes a relatively rigid proximal section 304 and a deflectable section 306 as has been discussed elsewhere herein. The lateral flexibility of distal section 306 may be accomplished in any of a variety of ways, such as by the provision of a plurality of transverse chevrons or slots 308. Slots 308 may be machined or laser cut into appropriate tube stock, such as stainless steel or any of a variety of rigid polymers.

The slots 308 oppose a column strength element such as an axially extending spine 310, for resisting axial elongation or compression of the device. A pull wire 312 axially moveably extends throughout the length of the tubular body, and is secured with respect to the tubular body distally of the transverse slots 308. The proximal end of the pull wire is operatively connected to a control on a proximal handpiece or manifold. The control may be any of a variety of structures, such as a lever, trigger, slider switch or rotatable thumb wheel or control knob. Axial proximal traction (or distal advance) of the pull wire 312 with respect to the tubular body causes a lateral deflection of the distal steering section 306, by axial compression or expansion of the transverse slots 308 relative to the spine 310.

A distal aperture 314 is in communication via a central lumen 316 with the proximal end of the steerable injector 300. Any of a variety of tip configurations may be used such as those disclosed elsewhere herein. The proximal end of the central lumen 316 may be provided with a luer connector, or other connection port to enable connection to a source of media such as bone cement to be infused. In the illustrated embodiment, the aperture 314 faces distally from the steerable injector 302, although other exit angles may be used as will be discussed below.

The steerable injector 300 is optionally provided with a cavity forming element 320, such as an inflatable balloon 322. In the illustrated embodiment, the inflatable balloon 322 is positioned in the vicinity of the steerable distal section 306. Preferably, the axial length of a distal leading segment 307 is minimized, so that the balloon 322 is relatively close to the distal end of the steerable injector 300. In this embodiment, the plurality of transverse slots 308 are preferably occluded, to prevent inflation media from escaping into the central lumen 316 or bone cement or other injectable media from escaping into the balloon 322. Occlusion of the transverse slots 308 may be accomplished in any of variety of ways: i) by positioning a thin tubular membrane coaxially about the exterior surface of the tubular body, and ii) heat shrinking or otherwise securing the membrane across the openings. Any of a variety of heat shrinkable polymeric sleeves, comprising high density polyethylene or other materials, is well known in the catheter arts. Alternatively, a tubular liner may be provided within the central lumen 316, to isolate the central lumen from the transverse slots 308.

The balloon 322 is secured at a distal neck 309 to the leading segment 307 as is understood in the balloon catheter arts. The distal neck 309 may extend distally from the balloon, as illustrated, or may invert and extend proximally along the tubular body. In either event, the distal neck 309 of the balloon 322 is preferably provided with an annular seal 324 either directly to the tubular body 301 or to a polymeric liner positioned concentrically about the tubular body, depending upon the particular device design. This will provide an isolated chamber within balloon 322, which is in fluid communication with a proximal source of inflation media by way of an inflation lumen 326.

In the illustrated embodiment, the balloon 322 is provided with an elongate tubular proximal neck which extends throughout the length of the steerable injector 300, to a proximal port or other site for connection to a source of inflation media. This part can be blow molded within a capture tube as is well understood in the balloon catheter arts, to produce a one piece configuration. Alternatively, the balloon can be separately formed and bonded to a tubular sleeve. During assembly, the proximal neck or outer sleeve 328 may conveniently be proximally slipped over the tubular body 301, and secured thereto, as will be appreciated by those of skill in the catheter manufacturing arts.

Referring to FIG. 16C, the inflation lumen 326 may occupy an annular space between the outer sleeve 328 and the tubular body 301. This may be accomplished by sizing the inside dimension of the outer sleeve 328 slightly larger than the outside dimension of the tubular body 301, by an amount sufficient to enable the desired inflation flow rate as will be understood in the art. Alternatively, referring to FIG. 16D, a discrete inflation lumen 326 may be provided while the remainder of the outer sleeve 328 is bonded or snuggly fit against the tubular body 301. This may be accomplished by positioning an elongate mandrel (not illustrated) between the outer sleeve 328 and the tubular body 301, and heat shrinking or otherwise reducing the outer sleeve 328, thereafter removing the mandrel to leave the discrete inflation lumen 326 in place. In another embodiment, a cross-section of a catheter with a balloon having an inflation lumen 326 with outer layer 350 coextensive with the outer surface of the balloon coaxial with sleeve 328 and tubular body 301 is shown in FIG. 16E. FIG. 16F illustrates a cross-section of another embodiment with an inflation lumen 326 external to the tubular body 301. FIG. 16G illustrates a cross-section of another embodiment with an inflation lumen 326 with a lumen internal to the tubular body 301. In some embodiments, the internal inflation lumen 326 can be integrally formed with the tubular body 301 as shown. Alternatively, any of a variety of other inflation lumen 326 configurations can be used.

In some embodiments, the cavity-creating element could include a reinforcing layer that may be, for example, woven, wrapped or braided (collectively a “filament” layer), for example, over the liner of a balloon. The filament layer can advantageously protect the balloon from damage while in the working space, for example from jagged cancellous bone fragments within the interior of the vertebral body. The filament layer can also significantly elevated the burst pressure of the balloon, such that it exceeds about 20 ATM, in some embodiments exceeds about 25 ATM, and in a preferred embodiment, is at least about 30 ATM.

The filament layer can also be configured to control the compliance of the balloon depending on the desired clinical result, either symmetrically or, if the filaments are asymmetric, to constrain expansion of the balloon in one or more directions. In some embodiments, the balloon can be said to have a first compliance value when inflated to a first volume at a given first pressure when the balloon expands without being mechanically constrained by the constraining element such as the filament layer. The balloon can have a second compliance value when further inflated to a second volume (greater than the first volume) at a given second pressure (greater than the first pressure) when the balloon expands while being mechanically constrained by the constraining element. The second compliance value is, in some embodiments, less than the first compliance value due to the effect of the constraining element on the balloon. The second compliance value can be, for example, at least about 5%, 10%, 15%, 20%, 25%, 30%, 40%, 50%, 60%, or 70% less than the first compliance value. In other embodiments, the second compliance value can be, for example, no more than about 70%, 60%, 50%, 40%, 30%, 25%, 20%, 15%, 10%, or 5% less than the first compliance value. In embodiments with a plurality of braided layers, the balloon could have an additional third, fourth, etc. progressively lower compliance values.

FIG. 16H schematically illustrates a vertebroplasty catheter 300 with a cavity creation element, namely a balloon 322 with a filament layer 340 carried by the balloon. FIG. 16I illustrates a cross-section of the filament reinforced balloon 322 through line 16I-16I of FIG. 16H, with filaments 340 surrounding the sidewall 350 of the balloon 322. FIG. 16J illustrates a cross-section of an alternative embodiment with filaments 340 over balloon sidewall 350 and also another layer 342 exterior to the braided layer 340. Other features have not been illustrated in FIGS. 16I and 16J for clarity. The exterior layer 342 could be made of, for example, a material discussed with respect to polymeric sleeve construction noted above, nylon, urethane, PET, or a thermoplastic. In some embodiments, there may be multiple layers, such as made of a polymer, exterior to the filament layer 340 and/or multiple liner layers interior to the filament 340, as well as multiple braided or other filament layers between or amongst the various layers. In some embodiments, the filament 340 is co-molded within a wall 350 of the balloon 322 itself.

The filament 340 may comprise any of a variety of metallic ribbons, although wire-based braids could also be used. In some embodiments, the ribbons can be made at least in part of wires in braids or made of strips of a shape memory material such as Nitinol or Elgiloy, or alternatively stainless steel, such as AISI 303, 308, 310, and 311. When using a braid 340 containing some amount of a super-elastic alloy, an additional step may be desirable in some embodiments to preserve the shape of the stiffening braid 340. For instance, with a Cr-containing Ni/Ti superelastic alloy which has been rolled into 1 mm×4 mm ribbons and formed into a 16-member braid 340, some heat treatment is desirable. The braid 340 may be placed onto a, e.g., metallic, mandrel of an appropriate size and then heated to a temperature of 600 degrees Fahrenheit to 750 degrees Fahrenheit for a few minutes, to set the appropriate shape. After the heat treatment step is completed, the braid 340 retains its shape and the alloy retains its super-elastic properties.

In some embodiments, metallic ribbons can be any of a variety of dimensions, including between about 0.25 mm and 3.5 mm in thickness and 1.0 mm and 5.0 mm in width. Ribbons can include elongated cross-sections such as a rectangle, oval, or semi-oval. When used as ribbons, these cross-sections could have an aspect ratio of thickness-width of at least 0.5 in some embodiments.

In some embodiments, the braid 340 may include a minor amount of fibrous materials, both synthetic and natural, may also be used. In certain applications, particularly in smaller diameter catheter sections, more malleable metals and alloys, e.g., bold, platinum, palladium, rhodium, etc., can be used. A platinum alloy with a few percent of tungsten is sometimes could be used partially because of its radio-opacity.

Nonmetallic ribbons or wires can also be used, including, for example, materials such as those made of polyaramides (Kevlar), polyethylene terephthalate (Dacron), polyamids (nylons), polyimide carbon fibers, or a shape memory polymer.

In some embodiments, the braids 340 can be made using commercial tubular braiders. The term “braid” when used herein includes tubular constructions in which the wires or ribbons making up the construction are woven in an in-and-out fashion as they cross, so as to form a tubular member defining a single lumen. The braid members may be woven in such a fashion that 2-4 braid members are woven together in a single weaving path, although single-strand weaving paths can also be used. In some embodiments, the braid 340 has a nominal pitch angle of 45 degrees. Other braid angles, e.g., from 20 degrees to 60 degrees could also be used.

In some embodiments, the cavity creation element includes two or more coaxial balloons, including an inner balloon 322 and an outer balloon 370 as illustrated schematically in FIG. 16O. Inner balloon 322 can be oriented in a first direction, such as more axially, while outer balloon 370 is oriented in a second direction, such as more radially. Balloon wall orientation, such as by stretching, is well understood in the art. The coaxial balloon configuration advantageously provides improved strength and burst resistance while minimizing the wall thickness of each balloon. Thus, two or more relatively thin-walled balloons can be utilized rather than a single thick-walled balloon to achieve both higher burst pressure and lower crossing profile. FIG. 16P illustrates a schematic cross-section of a section of the inner balloon wall 322 and outer balloon wall 370 that can be separated by a slip plane 372 that may have a friction-reducing lubricious coating or the like. In some embodiments, two, three, four, or more coaxially arranged balloons can be used in the same fashion. In some embodiments, one or more coaxial balloons is interspersed or integrated with one or more braided or other filament layers as described above. In some embodiments, each balloon could have a thickness of between about 0.0005 inches to 0.008 inches, or between about 0.001 inches to about 0.005 inches in other embodiments.

In some embodiments, the cavity creation element could be asymmetrical, for example, as with the balloon 344 offset from the longitudinal axis of the tubular body 301 illustrated schematically in FIG. 16K. Such a balloon configuration can be advantageous, for example, if the vertebral fracture is generally more anterior, so that the balloon 344 can be positioned to expand away from the anterior area to reduce the risk of balloon expansion causing a rupture all the way through the cortical bone of the vertebrae. A cross-sectional schematic view through the inflated offset balloon 344 is illustrated in FIG. 16L, also illustrating the tubular body 301. Other components such as guidewire 312 have been omitted for clarity purposes. In some embodiments, various balloons as described in FIGS. 1-20 and the accompanying disclosure of U.S. Pat. No. 6,066,154 to Reiley et al., which is hereby incorporated by reference in its entirety can also be used in connection with the injector 300 described herein. A schematic illustration of an offset balloon 344 on the catheter 300 when the distal segment 306 is deflected is illustrated in FIG. 16M.

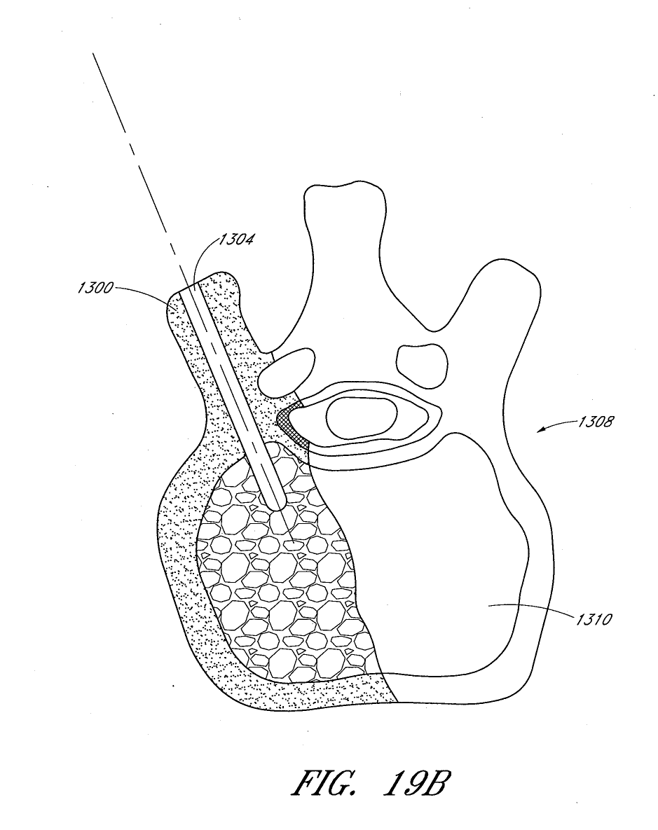

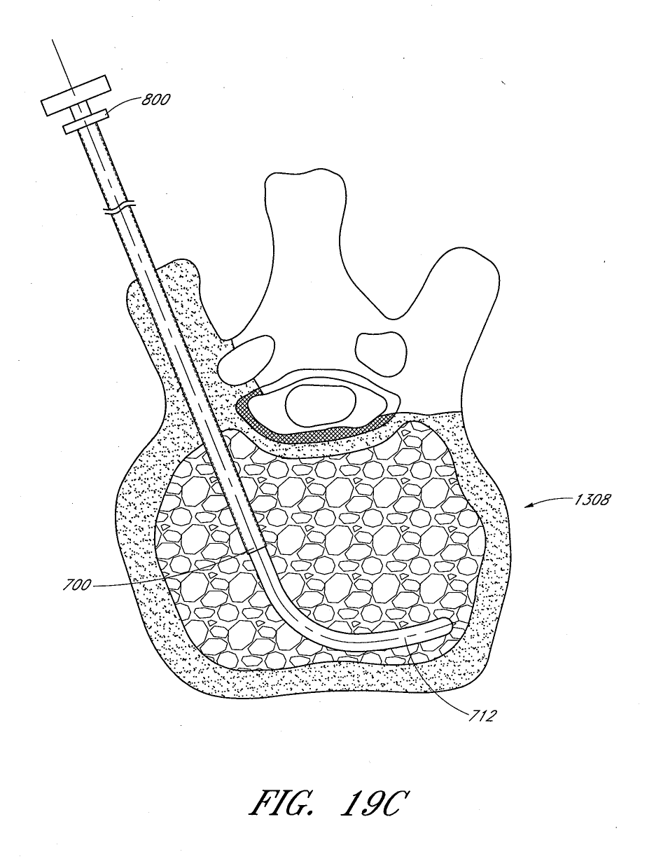

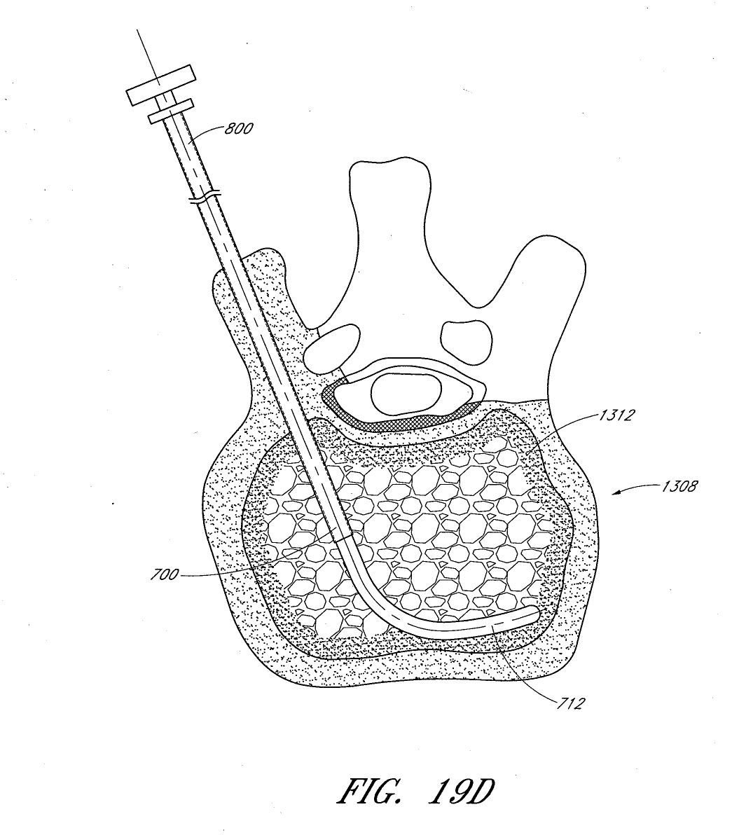

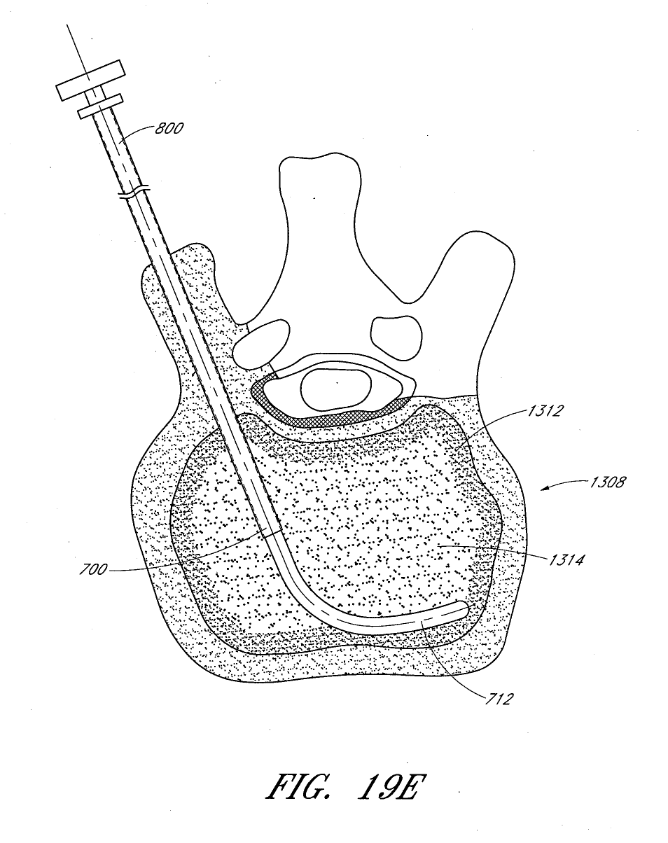

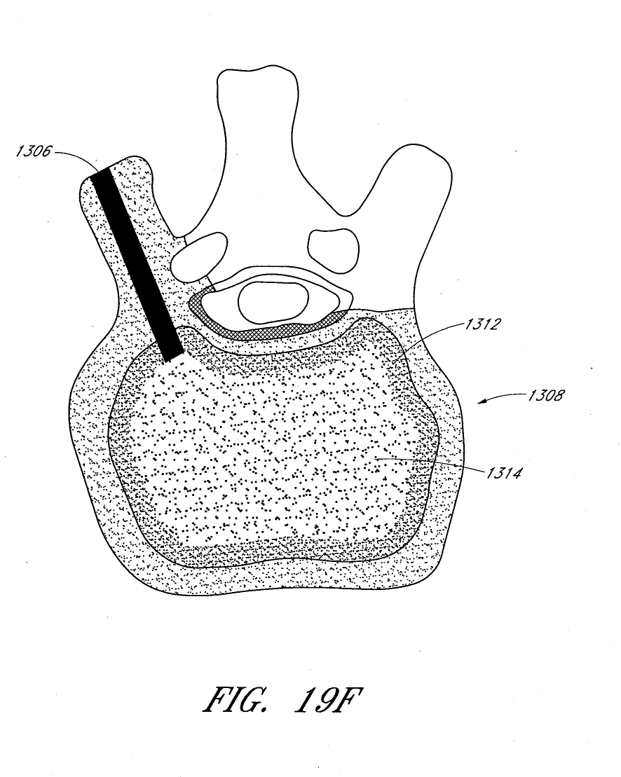

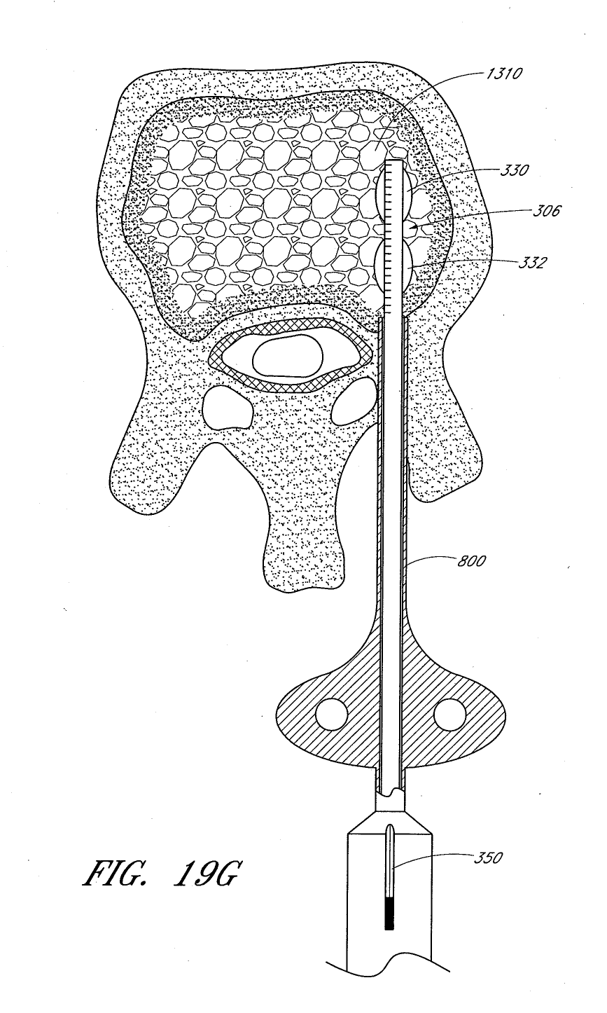

Referring to FIGS. 17A and 17B, there is illustrated an alternative embodiment in which the distal aperture 314 is provided on a side wall of the tubular body. One or two or three or more distal apertures 314 may be provided in any of the embodiments disclosed herein, depending upon the desired clinical performance. In the illustrated embodiment, the distal aperture 314 is provided on the inside radius of curvature of the steerable section 306, as illustrated in FIG. 17B. The aperture 314 may alternatively be provided on the opposite, outside radius of curvature, depending upon the desired clinical performance.

As a further alternative, the distal aperture or apertures 314 may be provided in any of a variety of configurations on a distal cap or tip, adapted to be secured to the tubular body.

In some embodiments, it may be advantageous to have multiple cavity-creation elements on a steerable injector in order to, for example, more quickly and efficiently move sclerotic cancellous bone to better facilitate cavity formation and the subsequent introduction of cement media. Referring to FIGS. 17C and 17D, there is an illustrated another embodiment of a steerable injector with a plurality of cavity creation elements thereon schematically illustrated, such as at least two, three, four, or more cavity creation elements. The cavity creation elements can be, for example, a first balloon 330 and a second balloon 332 as shown. As illustrated, both the first balloon 330 and the second balloon 332 are positioned in the vicinity of the steerable distal section 306. In other embodiments, as illustrated in FIGS. 17G and 17H, the first balloon 330 is positioned in the vicinity of the steerable distal section 306 while the second balloon 332 is positioned more proximally on the more rigid proximal section 304. In still other embodiments, as illustrated in FIGS. 17I and 17J, the first balloon 330 is positioned in the vicinity of the steerable distal section 306 while the second balloon 332 is positioned partially on the proximal section 304 and partially on the steerable distal section 306. In other embodiments, both the first balloon 330 and the second balloon 332 can be positioned in the vicinity of the proximal section 306.

In some embodiments, the first balloon 330 and the second balloon 332 share a common inflation lumen 326 (such as illustrated in FIG. 16C or D) and thus can be simultaneously inflatable from a common source of inflation media. In other embodiments, the first balloon 330 and the second balloon 332 have separate respective first inflation lumen 326 and second inflation lumen 327 and thus can be inflated according to the desired clinical result, e.g., simultaneously or the second balloon 332 inflated before or after the first balloon 330. FIGS. 17E and 17F are alternative cross sectional views showing different inflation lumen configurations. As illustrated in FIG. 17E, in some embodiments the first inflation lumen 328 can be positioned concentrically around the second inflation lumen 329, both of which can occupy annular spaces between the outer sleeve 328 and the tubular body 301. FIG. 17F illustrates an alternative embodiment where first 326 and second 327 discrete inflation lumens may be provided while the remainder of the outer sleeve 328 is bonded or snuggly fit against the tubular body 301.

The first balloon 330 and the second balloon 332 can have substantially the same properties or differing properties, such as thickness, material, inflation diameter, burst strength, compliance, or symmetry (or lack thereof) depending on the desired clinical result. In some embodiments, the distal aperture 314 could be distally facing, positioned on a side wall, or on an inclined surface; or 2, 3, 4, 5, or more apertures could be presented as previously described. Furthermore, while the aperture 314 is illustrated in FIGS. 17C-17D, and 17G-17J as positioned on the distal end of the catheter 300 as being distal to both first balloon 330 and second balloon 332 in some embodiments the aperture 314 or additional aperture(s) can be positioned in between first balloon 330 and second balloon 332 and/or proximal to second balloon 332. In embodiments with one or more cavity creating elements having multiple apertures, the apertures could be fluidly communicate with each other, or be fluidly isolated in other embodiments.

The steerable injection systems described above are preferably used in conjunction with a mixing and dispensing pump for use with a multi-component cement. In some embodiments, a cement dispensing pump is a hand-held device having an interface such as a tray or chamber for receiving one or more cartridges. In one embodiment, the pump is configured to removably receive a double-barreled cartridge for simultaneously dispensing first and second bone cement components. The system additionally includes a mixing chamber, for mixing the components sufficiently and reproducibly to fully automate the mixing and dispensing process within a closed system.

Bone cement components have conventionally been mixed, such as by hand, e.g., in mixing bowls in the operating room, which can be a time-consuming and unelegant process. The devices disclosed herein may be used with conventional bone cement formulations, such as manually mixed liquid-powder PMMA formulations. Alternatively, the use of a closed mixing device such as a double-barreled dispensing pump as disclosed herein is highly advantageous in reducing bone cement preparation time, preventing escape of fumes or ingredients, ensuring that premature cement curing does not occur (i.e., the components are mixed immediately prior to delivery into the body), and ensuring adequate mixing of components.

Two separate chambers contain respective materials to be mixed in a specific ratio. Manual dispensing (e.g., rotating a knob or squeezing a handle) forces both materials into a mixing nozzle, which may be a spiral mixing chamber within or in communication with a nozzle. In the spiral mixing nozzle, all or substantially all mixing preferably occurs prior to the bone cement entering the steerable injection needle and, subsequently, into the vertebra. The cement dispensing hand pump may be attached to the steerable injection needle permanently or removably via a connector, such as slip-ring Luer fittings. A wide range of dispensing pumps can be modified for use with the present invention, including dispensing pumps described in, for example, U.S. Pat. Nos. 5,184,757, 5,535,922, 6,484,904, and Patent Publication No. 2007/0114248, all of which are incorporated by reference in their entirety.

Currently favored bone cement compositions are normally stored as two separate components or precursors, for mixing at the clinical site shortly prior to implantation. As has been described above, mixing of the bone cement components has traditionally been accomplished manually, such as by expressing the components into a mixing bowl in or near the operating room. In accordance with the present invention, the bone cement components may be transmitted from their storage and/or shipping containers, into a mixing chamber, and into the patient, all within a closed system. For this purpose, the system of the present invention includes at least one mixing chamber positioned in the flow path between the bone cement component container and the distal opening on the bone cement injection needle. This permits uniform and automated or semi-automated mixing of the bone cement precursors, within a closed system, and thus not exposing any of the components or the mixing process at the clinical site.

Thus, the mixing chamber may be formed as a part of the cartridge, may be positioned downstream from the cartridge, such as in-between the cartridge and the proximal manifold on the injection needle, or within the proximal manifold on the injection needle or the injection needle itself, depending upon the desired performance of the device. The mixing chamber may be a discrete component which may be removably or permanently coupled in series flow communication with the other components of the invention, or may be integrally formed within any of the foregoing components.

In general, the mixing chamber includes an influent flow path for accommodating at least two bone cement components. The first and second incoming flow paths are combined, and mixing structures for facilitating mixing of the components are provided. This may include any of a variety of structures, such as a helical flow path, baffles and or additional turbulence inducing structures.

Tables 1-2 below depict the contents and concentrations of one exemplary embodiment of bone cement precursors. Chambers 1A and 1B contain precursors for a first cement composition for distribution around the periphery of the formed in place vertebral body implant with a higher particle concentration to promote osteoinduction, as discussed previously in the application. Chambers 2A and 2B contain precursors for a second cement composition for expression more centrally within the implanted mass within the vertebral body, for stability and crack arresting, as discussed previously in the application.

One of ordinary skill in the art will recognize that a wide variety of chamber or cartridge configurations, and bone cements, can be used with the present injection system. For example, in one embodiment, a first cartridge includes pre-polymerized PMMA and a polymerization catalyst, while a second cartridge includes a liquid monomer of MMA as is common with some conventional bone cement formulations.

In some embodiments, the contents of two cartridges can be combined into a single cartridge having multiple (e.g., four) chambers. Chambers may be separated by a frangible membrane (e.g., 1A and 2A in a first cartridge and 1B and 2B in a second cartridge, each component separated by the frangible membrane or other pierceable or removable barrier). In other embodiments, contents of the below cartridges can be manually pre-mixed and loaded into the input port of the injection system without the use of a cement mixing dispenser.

TABLE 1
Chamber 1A

| | |
|---|---|
| Methyl methacrylate (balance) | Hydroquinone (~75 ppm)(stabilizer) |
| N,N-dimethyl-p-toluidine (~0.9%) | Sterile bone particles (≧35 wt. %) |
| (catalyst for polymerization) | |
| Barium sulfate | |
| (~20 wt. %) (radio-opacifier) | |

Chamber 1B

| | |
|---|---|
| Benzoyl peroxide (~2%) | Physiological saline or poppy seed oil |
| (activator for polymerization) | (balance) |

TABLE 2
Chamber 2A

| | |
|---|---|
| Methyl methacrylate (balance) | Hydroquinone (~75 ppm)(stabilizer) |
| N,N-dimethyl-p-toluidine (~0.9%) | Sterile bone particles (~30 wt. %) |
| (catalyst for polymerization) | |
| Barium sulfate | |
| (~20 wt. %) (radio-opacifier) | |

Chamber 2B

| | |
|---|---|
| Benzoyl peroxide (~2%) | Physiological saline or poppy seed oil |
| (activator for polymerization) | (balance) |

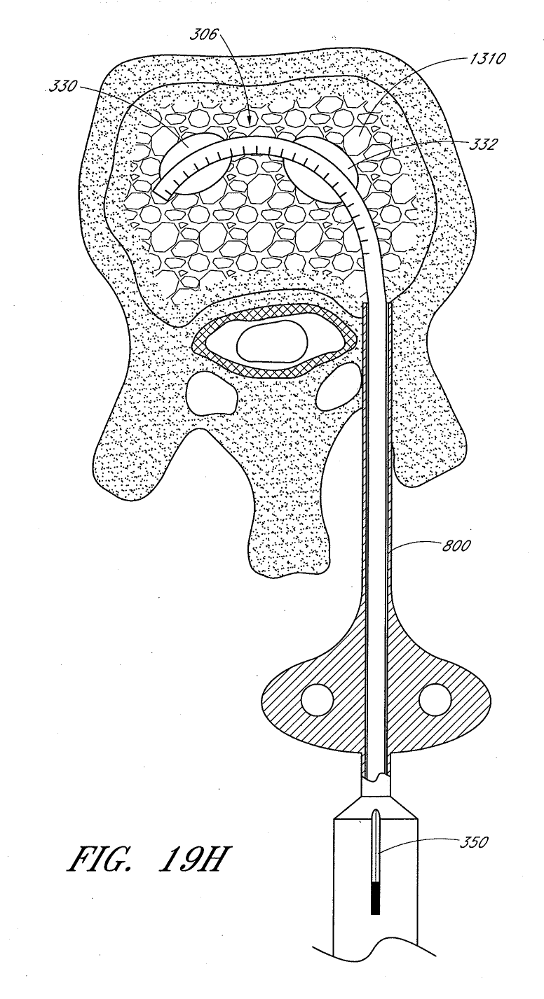

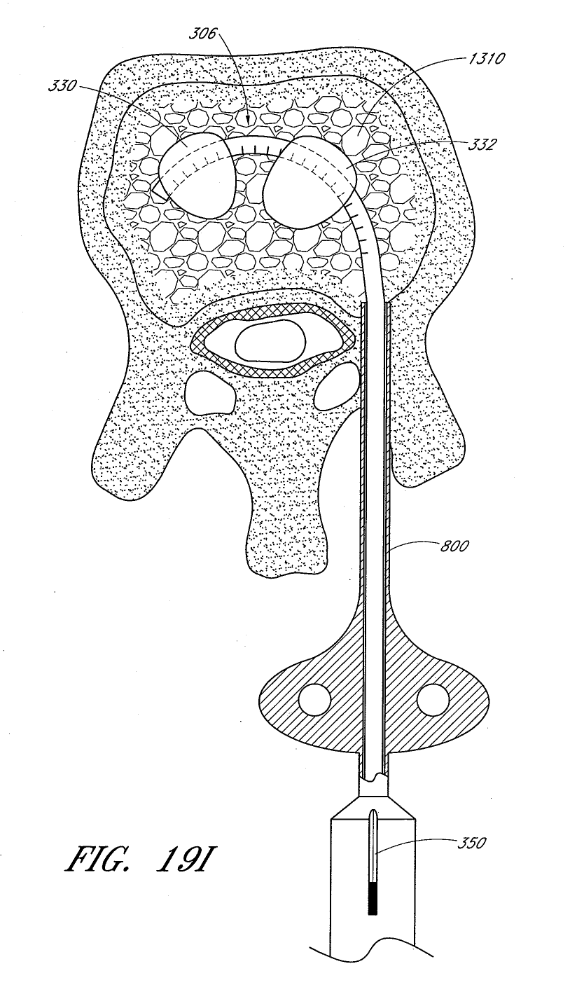

As illustrated in FIGS. 18A and 18B, in one embodiment, a system or kit for implanting bone cement includes at least some of the following components: a stylet configured to perforate a hole into the pedicle of the vertebral body; an introducer cannula 800 for providing an access pathway to the treatment site, a steerable injection needle 700 to deliver bone cement to a desired location, and, a cement dispensing pump 910 preferably configured to accommodate one or two or more dual chamber cartridges 1200 as well as a mixing nozzle 995.

The stylet may have a diameter of between about 0.030″ to 0.300″, 0.050″ to about 0.200″ and preferably about 0.100″ in some embodiments. The introducer cannula 800 is between about 8-14 gauge, preferably between about 10-12 gauge, more preferably 11 gauge in some embodiments. The introducer cannula 800, which may be made of any appropriate material, such as stainless steel (e.g., 304 stainless steel) may have a maximum working length of no more than about 12″, 8″, or 6″ in some embodiments. One or two or more bone cement cartridges, each having one or two or more chambers, may also be provided. Various other details of the components have been described above in the application.

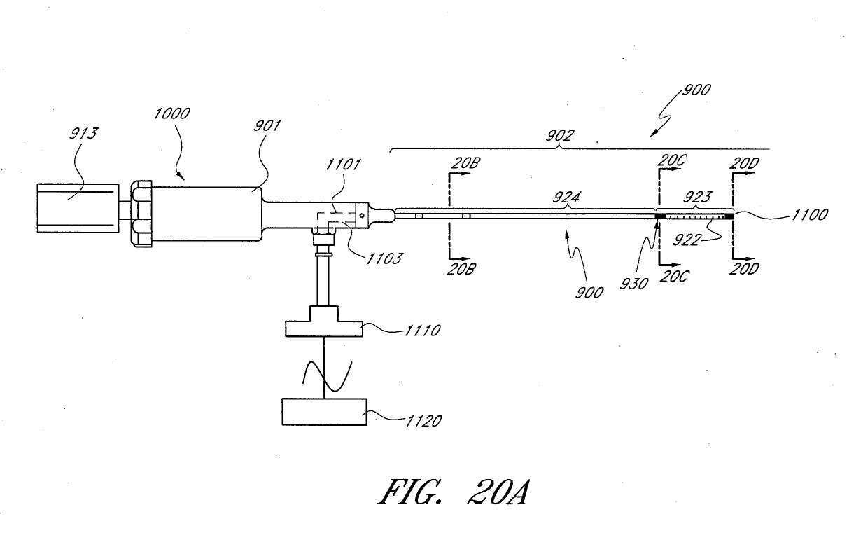

One embodiment of a method for delivering bone cement into a vertebral body is now described, and illustrated in FIGS. 19A-F. The method involves the general concept of vertebroplasty and kyphoplasty in which a collapsed or weakened vertebra is stabilized by injecting bone cement into cancellous bone.

The cement implantation procedure is designed for uni-transpedicular access and generally requires either a local anesthetic or short-duration general anesthetic for minimally invasive surgery. Once the area of the spine is anesthetized, as shown in FIGS. 19A-B, the physician inserts a stylet 1302 to perforate a lumen 1304 into the pedicle wall 1300 of the vertebra 1308 to gain access to the interior of the vertebral body 1310. As illustrated in FIG. 19C, the introducer cannula 800 is then inserted through the lumen 1304 for bone access as well as acting as the guide for the steerable injection needle 700. The introducer cannula 800 is sized to allow physicians to perform vertebroplasty or kyphoplasty on vertebrae with small pedicles 1300 such as the thoracic vertebra (e.g., T5) as well as larger vertebrae. In addition, this system and method is advantageously designed to allow uni-transpedicular access as opposed to bi-pedicular access, resulting in a less invasive surgical procedure.

Once bone access has been achieved, as shown in FIG. 19C the steerable injection needle 700 such as any of the devices described above can be inserted through the introducer cannula 800 and into the vertebra 1308. The entire or a portion of the cancellous bony interior 1310 of the target vertebral body may be accessed using the steerable injection needle 800. To avoid the possibility of any extravasated cement from coming in contact with critical structures such as the spinal cord and or the spinal roots, only the anterior ⅔ of the cancellous bone space is injected with cement in some embodiments. The distal end 712 of the needle 700 can be laterally deflected, rotated, and/or proximally retracted or distally advanced to position the bone cement effluent port at any desired site as previously described in the application. The radius can be adjusted by means of an adjustment control, such as a knob on the proximal end of the device as previously described.

The actual injection procedure may utilize either one or two basic steps. In a one step procedure, a homogenous bone cement is introduced as is done in conventional vertebroplasty. The first step in the two step injection involves injection of a small quantity of PMMA with more than about 35%, e.g., 60% particles such as inorganic bone particles onto the periphery of the treatment site, i.e., next to the cortical bone of the vertebral body as shown in FIG. 19D. This first cement composite 1312 begins to harden rather quickly, forming a firm but still pliable shell, which is intended to minimize or prevent any bone marrow/PMMA content from being ejected through any venules or micro-fractures in the vertebral body wall. The second step in the procedure involves an injection of a bolus of a second formulation of PMMA with a smaller concentration such as approximately 30% inorganic bone particles (second cement composite 1314) to stabilize the remainder of the weakened, compressed cancellous bone, as illustrated in FIG. 19E.

Injection control for the first and second steps is provided by an approximately 2 mm inside diameter flexible introducer cannula 800 coupled to a bone cement injection pump (not shown) that is preferably hand-operated. Two separate cartridges containing respective bone cement and inorganic bone particle concentrations that are mixed in the 60% and 30% ratios are utilized to control inorganic bone particle to PMMA concentrations. The amount of the injectate is under the direct control of the surgeon or interventional radiologist by fluoroscopic observation. The introducer cannula 800 is slowly withdrawn from the cancellous space as the bolus begins to harden, thus preventing bone marrow/PMMA content from exiting the vertebral body 1308. The procedure concludes with the surgical incision being closed, for example, with bone void filler 1306 as shown in FIG. 19F. Both the high and low bone cement particle concentration cement composites 1312, 1314 harden after several minutes. In vitro and in vivo studies have shown that the 60% bone-particle impregnated bone cement hardens in 2-3 minutes and 30% bone-particle impregnated bone cement hardens between 4 to 10 minutes.

The foregoing method can alternatively be accomplished utilizing the combination steerable needle of FIG. 16A, having a cavity formation structure 320 thereon. Once the steerable injector 300 has been positioned as desired, such as either with deflection as illustrated in FIG. 19C, or linearly, the cavity forming element 320 is enlarged, such as by introducing inflation media under pressure into the inflatable balloon 322. The cavity forming element 320 is thereafter reduced in cross sectional configuration, such as by aspirating inflation media from the inflatable balloon 322 to produce a cavity in the adjacent cancellous bone. The steerable injector 300 may thereafter by proximally withdrawn by a small distance, to position the distal opening 314 in communication with the newly formed cavity. Bone cement or other media may thereafter be infused into the cavity, as will be appreciated by those skill in the art.

At any time in the process, whether utilizing an injection needle having a cavity formation element or not, the steerable injector may be proximally withdrawn or distally advanced, rotated, and inclined to a greater degree or advanced into its linear configuration, and further distally advanced or proximally retracted, to position the distal opening 314 at any desired site for infusion of additional bone cement or other media. More than one cavity, such as two, or three or more, may be sequentially created using the cavity formation element, as will be appreciated by those of skill in the art.

The aforementioned bone cement implant procedure process eliminates the need for the external mixing of PMMA powder with MMA monomer. This mixing process sometimes entraps air, bone marrow and blood in the dough, thus creating porosity in the hardened PMMA in the cancellous bone area. These pores weaken the PMMA. Direct mixing and hardening of the PMMA using an implant procedure such as the above eliminates this porosity since no air, bone marrow or blood are entrapped in the injectate. This, too, eliminates further weakening, loosening, or migration of the PMMA.

A method of using the steerable injection system described, for example, in FIGS. 17C-17D will now be described. Various components of the injector 300 are not illustrated for clarity purposes. The interior of the vertebral body 1310 can be first accessed via a unipedicular approach as described and illustrated in connection with FIGS. 19A-B. Next, the steerable injector 300 having first balloon 330 and second balloon 332 thereon is inserted through an introducer 800 into the interior of the vertebral body 1310 with the distal deflectable section 306 in a relatively straightened configuration, as shown schematically in FIG. 19G. In some embodiments, the injector 300 also has a retractable outer sheath 340 actuatable by a control 350 on the handpiece 360 to protect the balloons 330, 332 from damage during introduction of the injector 300 into the interior of the vertebral body 1310. The injector 300 can then be laterally deflected, rotated, and or proximally retracted or distally advanced to position the injector at any desired site as previously described in the application, and illustrated schematically in FIG. 19H. The radius can be adjusted by means of an adjustment control, such as a knob on the proximal end of the device as previously described. The first balloon 330 and second balloon 332 can then be inflated simultaneously as illustrated in FIG. 19I or sequentially as previously described. In some embodiments, only one of the balloons may need to be inflated depending on the size of the cavity desired to be created. Injection of the cement media can proceed at any desired time as previously described, such as, for example, following deflation of one or both balloons.

### Steerable, Curvable Energy Delivery Catheter for Vertebroplasty

Also disclosed herein is a steerable, curvable catheter that can be used to ablate tissue, such as bone, in a wide variety of applications including vertebroplasty or kyphoplasty. In some embodiments, prior to or concurrent with an orthopedic procedure such as vertebroplasty or kyphoplasty, it may be advantageous to remove bone or other tissue, such as sclerotic cancellous bone, in order to facilitate adequate filling of the interior of a vertebral body with bone cement or to create or enhance cavity formation in a kyphoplasty procedure. Systems, devices, and methods to facilitate removal of such tissue such as sclerotic cancellous bone will now be described. While one embodiment illustrated is a steerable, curvable dipole RF ablation catheter, catheters with one or more monopolar RF electrodes or catheters with a tip or other area(s) configured to ablate tissue with other energy modalities are also within the scope of the invention. For example, other types of energy that can be used to ablate tissue include laser, ultrasound such as focused ultrasound or high intensity focused ultrasound (HIFU), microwave, infrared, visible, or ultraviolet light energy, electric field energy, magnetic field energy, cryoablation, combinations of the foregoing, or other modalities. For some forms of energy, the energy can be launched from a source carried by the distal end of the catheter, such as, for example, ultrasound transducers, microwave coil arrays, laser light sources, and others as will be understood in the art. For the same, or other energy forms, the energy source may be coupled to the proximal end of the catheter and the energy propagated distally through the catheter to an energy interface at the distal end of the catheter. Energy may be propagated along any appropriate conduit or circuit, such as fiber optics, conductive wires, one or more lumens (e.g., for cryogenic media) or others as appropriate for the energy source.

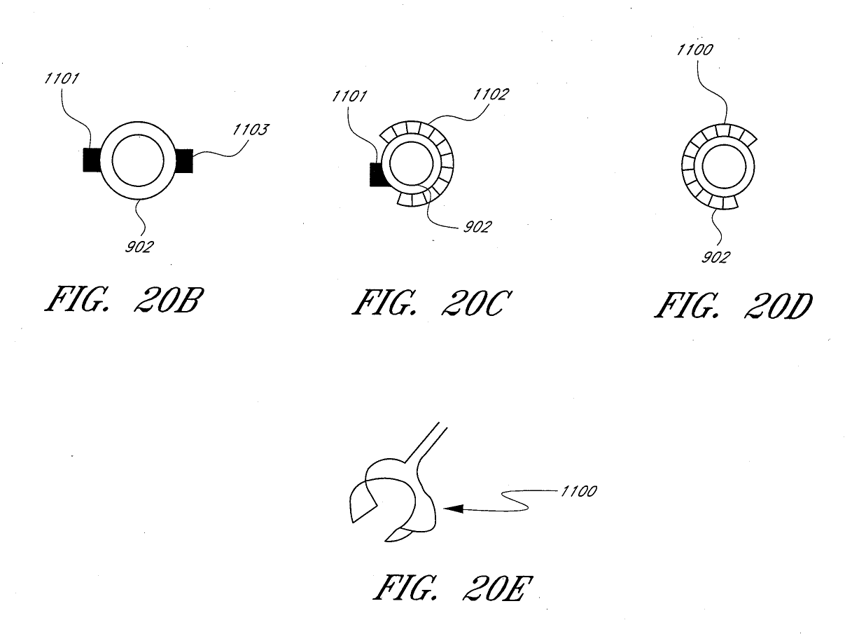

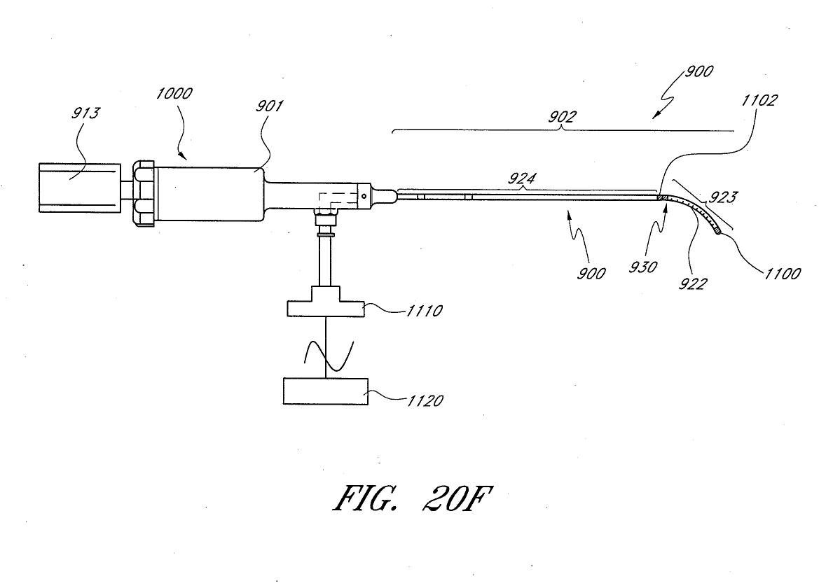

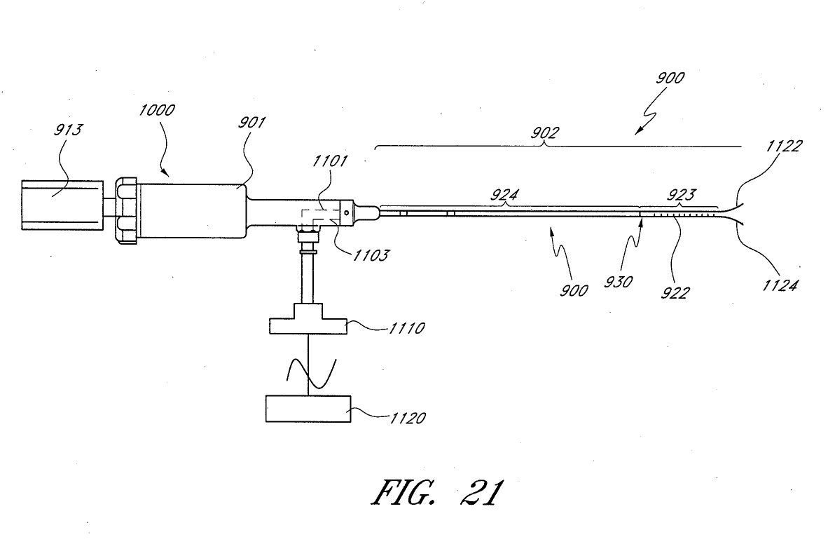

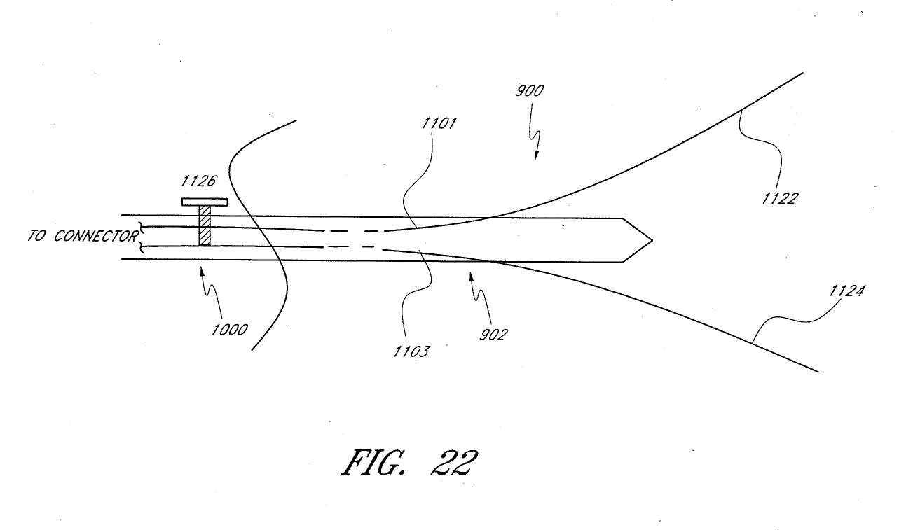

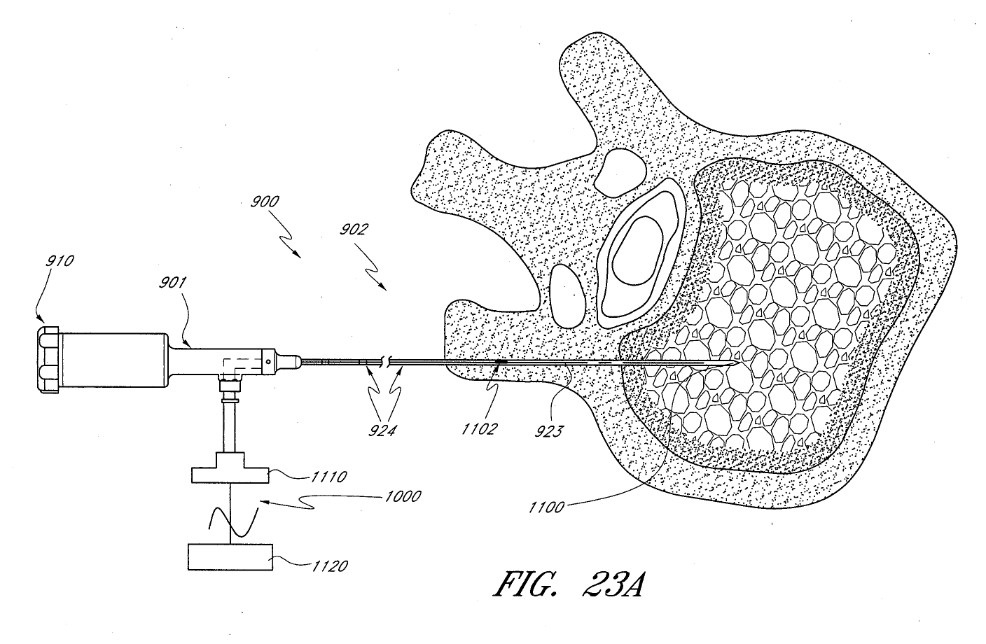

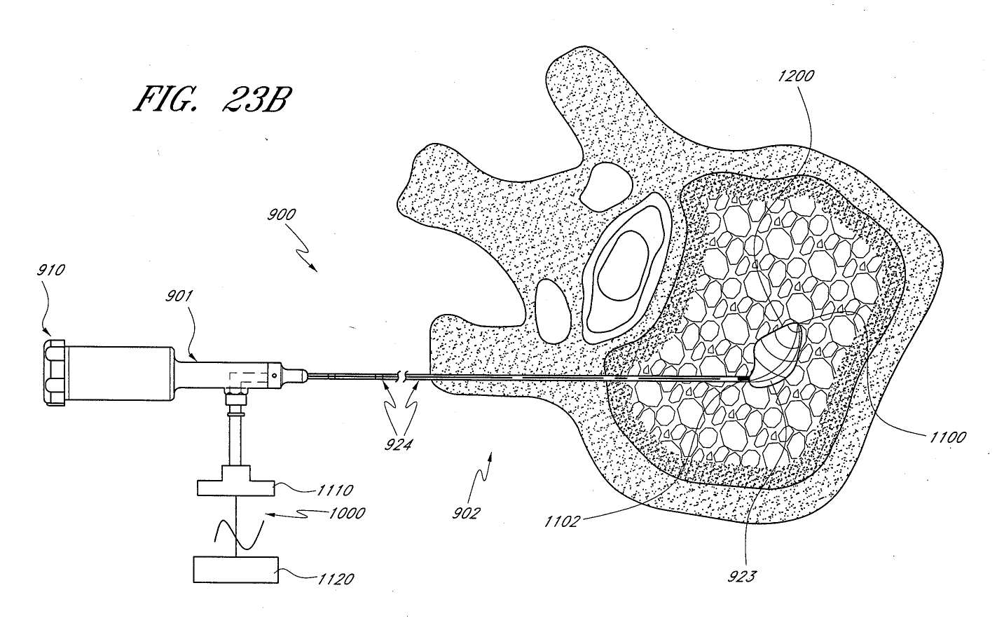

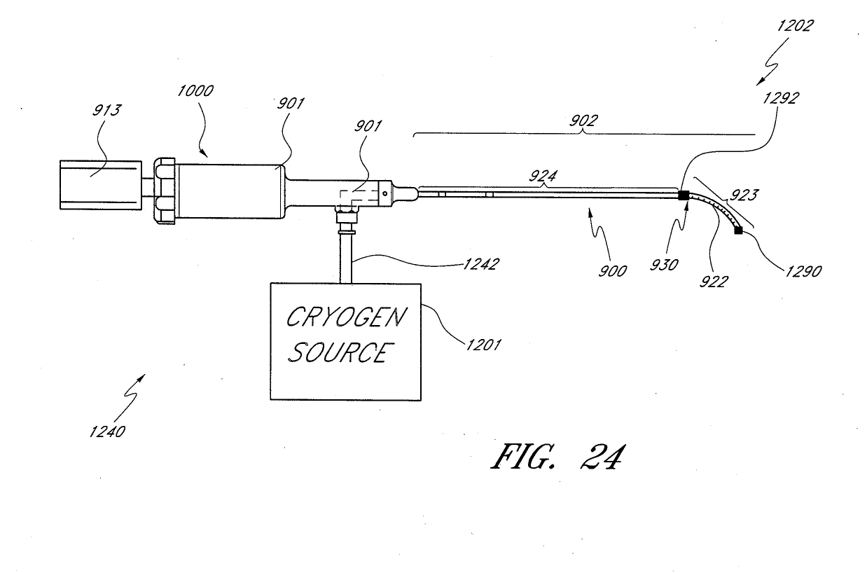

Referring to FIG. 20A, disclosed is a perspective view of a steerable, curvable ablation catheter in accordance with one embodiment of the invention. Catheter 900 has an elongate body including a proximal handle portion 1000 and an elongate member 902. One or two or more ablation or energy delivery elements 1100, 1102 such as RF electrodes (also referred to herein as RF antennas) can be operably connected to any portion of the elongate member 902, such as in the vicinity of distal segment 923 in order to ablate a tissue such as cancellous bone. The energy delivery elements 1100, 1102 are connected to respective first energy delivery system and second energy delivery system 1103 which are in turn connected more proximally via connector 1110 to a power source such as an RF generator 1120. Connector 1110 can be a universal connector configured to connect to a wide range of available generators such that those available, for example, from AngioDynamics, Inc. (Queensbury, N.Y.) such as the RITA® system; or Integra Radionics (Plainsboro, N.J.).

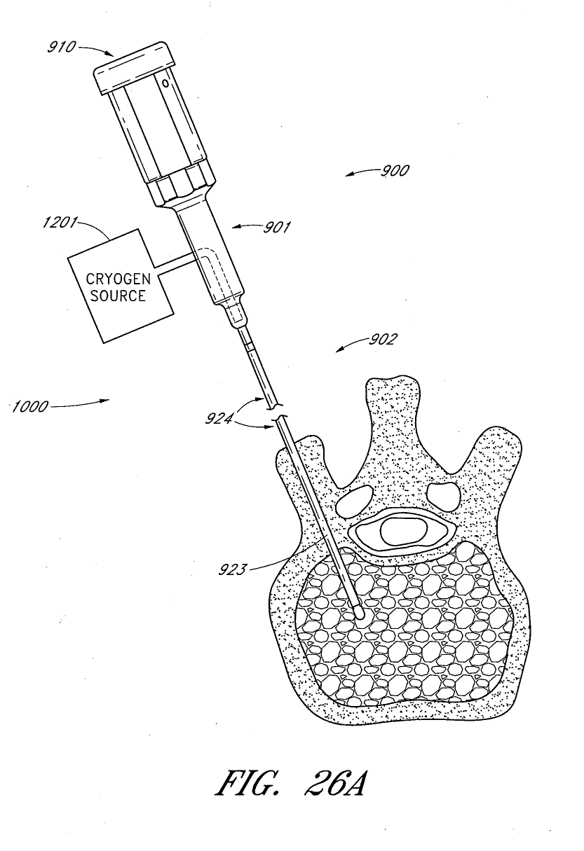

However, in other embodiments one or more energy delivery elements could be wirelessly activated via an external energy source. In some embodiments, the first RF electrode 1100 and the second RF electrode 1102 are separated by a distance of between about 0.1-10 cm, such as between about 0.5-5 cm, 1-3 cm, or about 2 cm in some embodiments depending on the desired area of treatment. The electrodes can be made of any appropriate material, such as a conductive, flexible corrosion resistant material such as copper, silver, or other metal and optionally coated with another metal such as gold or palladium. The antenna conduits 1101, 1103 can be shielded/insulated from the elongate tubular body 902 that conduits 1101, 1103 run along the outer diameter of by any appropriate material, such as, for example, a nonconductive coating or varnish. However, in other embodiments, one or more of the conduits 1101, 1103 run along the inside diameter of the tubular body 902, such as within a lumen configured to house the conduits 1101, 1103 therethrough. The distal elongate member 902 includes, in some embodiments, a relatively rigid proximal end or segment 924 and a distal deflectable end or segment 923 separated by a transition point 930 defining where the distal portion 923 is configured to be deflectable. The proximal segment 924 can be relatively straight and coaxial with the long axis of the proximal handle portion 1000. The distal segment 923 can be configured to be steerable and curvable through a working range via actuation of a control 901 on the handle 1000 that can be as described in detail supra in the application, such as, for example, at FIGS. 4-5, 9A-10C and the accompanying disclosure. As described elsewhere herein, the lateral flexibility of the distal section 923 may be accomplished in any of a variety of ways, such as by the provision of a plurality of transverse chevrons or slots 922. Slots 922 may be machined or laser cut into appropriate tube stock, such as stainless steel or any of a variety of rigid polymers. Slots 922 oppose a column strength element such as an axially extending spine, for resisting axial elongation or compression of the device. A deflection control 901 may be operably connected to one or more pullwires or other element configured to deflect the distal segment 923 through a working range as previously described, to deflect the catheter 900 as illustrated in FIG. 20F. The proximal handle portion 1000 can include a control 913 such as a simple on-off push-button control, switch, or the like or a rotary dial or other control that allows for power adjustment depending on the desired clinical result. In some embodiments, the proximal handle portion 1000 can also include an energy delivery control for operating the energy delivery elements. The energy delivery control can be used to determine the power level of the energy applied. In some embodiments, the energy delivery control comprises a retraction control 1126 (as shown in FIG. 22) such as a slider, dial, or other control operably connected to energy delivery conduit(s) and configured to retract energy delivery elements. In other embodiments, the energy delivery control is, for example, a foot pedal.

In some embodiments, the catheter includes one or more thermocouple wires that can determine the temperature at the treatment site. The system could include a feedback system that shuts down power when the temperature exceeds a certain predetermined parameter. In some embodiments, the system is configured to heat the targeted tissue for ablation to a temperature of between about 45 and 90 degrees Celsius, or at or at least about 40, 50, 60, 70, 80, 90, 100, 110, or more degrees Celsius in other embodiments. In other embodiments, the temperature may be no more than about 110, 100, 90, 80, 70, 60, or 50 degrees Celsius, or less. In some embodiments, when a monopolar system is used a passive electrode, return electrode or ground pad replaces one of the first electrode 1100 or second electrode 1102. Other elements of RF systems that can be used with the catheter 900 described herein can be found, for example, in U.S. Pat. No. 6,749,624 to Knowlton or U.S. Pat. Pub. No. 2009/0082762 to Ormsby et al., both of which are hereby incorporated by reference in their entirety. In some embodiments, the catheter can incorporate a cooling system such as a supply lumen and a return lumen for providing a liquid cooling circuit running in the vicinity of the energy delivery element in order to cool tissue before, during, and/or after application of energy.

Generator 1120 can operate at any appropriate frequency. For an RF generator, the frequency could be less than about 30 Mhz, such as between about 375-500 kHz or between about 430-490 kHz or between about 460-480 kHz in some embodiments. The generator could have any appropriate power output depending on the desired clinical results, such as less than about 500 W, such as about 150 W, 200 W, or 250 W in some embodiments.

FIG. 20B is a cross-section through line 20B-20B of FIG. 20A, and illustrates first RF antenna wire 1101 and second RF antenna wire 1103 connected proximally via connector 1110 to an RF generator 1120 and distally to their respective RF electrode 1100 and 1102.

FIG. 20C is a more distal cross-section through line 20C-20C at the point on the catheter 900 through first antenna 1102. As illustrated, first antenna 1102 partially circumscribes the elongate catheter tube body 902 in the vicinity of the transition point 930, although in other embodiments the first antenna 1102 could be located more axially distally or proximally depending on the desired clinical result. In one embodiment as shown, first antenna 1102 partially circumscribes the outer diameter of the elongate catheter tube body 902 by about 350 degrees, leaving about 10 degrees for second antenna wire 1101 to continue running distally to the end of second antenna 1100. Shielding or insulation layer is not shown for clarity. This configuration advantageously allows antenna wires 1101, 1103 to run across the tube body 902 without crossing each other. However, first antenna 1102 could also fully circumscribe the tube body 902 and second antenna wire 1101 could run either over or under the first antenna 1102 separated by a shielding or insulation element. In other embodiments, an antenna 1100, 1102 could circumscribe the tubular body 902 by any other desired angle, such as less than about 300, 270, 240, 210, 180, 150, 120, 90, 60, or less degrees.

FIG. 20D is a still more distal cross-section through line 20D-20D at the point on the catheter 900 through second antenna 1100. Second antenna 1100 could fully circumscribe the distal end of the tubular body 902, or partially circumscribe the antenna 1100 at the same angle as the first antenna 1102, or at a lesser or greater angle depending on the desired clinical result. FIG. 20E is a perspective view of one embodiment of either antenna 1100 or second antenna 1102, although other antenna shapes and configurations are also within the scope of the invention. In some embodiments, the electrode itself can have a diameter of, for example between about 0.1 mm-2 cm, such as between about 1-10 mm, or between about 3-7 mm.

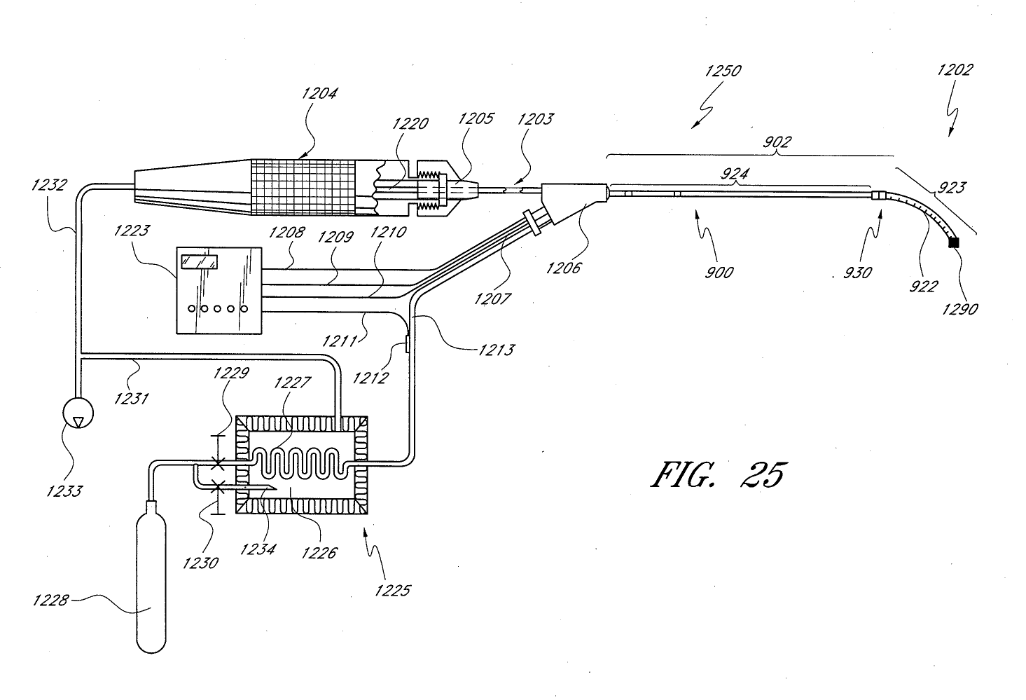

FIG. 21 illustrates an alternative embodiment of a catheter 900 with RF electrodes 1122, 1124 extending distally and radially outwardly from the distal end 923 of the tubular body 902 of the catheter 900. In such an embodiment, the antenna wires 1101, 1103 could run along the inside diameter (interior) of the tubular body 902 rather than the outside diameter (exterior). As previously noted, in a dipole RF system the electrodes 1122 could be separated by any desired distance depending on the amount of tissue to be treated, such as between about 1-3 cm, or about 2 cm in one embodiment. In some embodiments, deflection of the catheter can vary a distance between the electrodes 1122, 1124. In other embodiments such as a monopolar RF system or system employing another energy modality a single energy delivery element may just extend axially from the distal end 923 of the catheter 900 or be located at a more proximal location. In other embodiments, the catheter 900 may deploy 2, 3, 4, 5, or more energy delivery elements.

FIG. 22 illustrates schematically an embodiment of a catheter 900 similar to that as illustrated and described in connection with FIG. 21 with a retractable energy delivery element feature to protect the energy delivery elements while catheter 900 is being inserted or withdrawn from the body. As illustrated, catheter 900 has RF electrodes 1122, 1124 extending distally and radially outwardly from the distal end 923 of the tubular body 902 of the catheter 900. Proximal handle 1000 portion of catheter 900 can include an energy delivery element retraction control 1126 such as a slider, dial, or other control operably connected to energy delivery conduit(s) and configured to exert tension on wires 1101, 1103 to retract electrodes 1122, 1124. In other embodiments, alternatively a retractable sheath or outer catheter member can be used to protect the energy delivery elements.

In some embodiments, the steerable energy delivery vertebroplasty catheter 900 can be used to create a cavity in a vertebral body wherein various types of bone cements can be injected as described supra in the application as well as those disclosed in U.S. patent application Ser. No. 11/626,336, filed Jan. 23, 2007, the disclosure of which is hereby incorporated in its entirety herein by reference.

In some embodiments, this procedure for creating a cavity or otherwise preparing the bone for inserting one of the bone cements described above is performed by making an incision in an appropriate location and inserting the distal portion of the steerable vertebroplasty catheter 900 to the desired osteotomy site. A number of features of this device minimize the degree of invasiveness required to perform this procedure: While the relatively low-profile diameter of the tube 902 minimizes the size of the incision required to guide the device to this location, the ability to change the angle of actuation of the distal segment 923 in which energy delivery members are attached to gives the user significant flexibility in determining the surgical approach for the desired location for making the osteotomy. Once the operator has maneuvered the distal segment 923 of the steerable vertebroplasty catheter 900 to the desired osteotomy site, the operator is then able to activate the energy delivery member(s). The operator can create a cavity therein by activating the energy delivery features of the catheter 900 in the center of the vertebral body thereby ablating the diseased bone. This can be facilitated through the use of the deflectable distal portion 923 of the tube 902 that enables the user to change the angle of deflection without changing the angle of approach. In this manner, a relatively large cavity can advantageously be created within the bone with only a minimally invasive approach and single entry site. One embodiment of this procedure is depicted in FIGS. 23A-B, with schematic ablation zone 1200 illustrated in FIG. 23B.

Energy could be delivered to the target tissue for any appropriate time period depending on the desired clinical result. For example, in some embodiments tissue to be ablated could be heated to at least about 60, 70, 80, 90, or 100 degrees Celsius for less than about 5, 4, 3, 2, 1 minute or less. In some embodiments, tissue such as cortical or cancellous bone is heated to between about 90 to 110 degrees Celsius for between about 1 to 8 minutes, or between about 4 to 6 minutes. In some embodiments, the maximum current achievable to the vertebral body can be at or less than about 350 mA, 300 mA, 250 mA, or 200 mA.

Once a zone or cavity of sufficient size has been created within the vertebral body or other bone, the operator can then withdraw the steerable vertebroplasty ablation catheter 900 and introduce a device capable of injecting the desired bone cement or other compound into the cavity as described, for example, elsewhere in the application. As described below, in some embodiments, the cavity creation/ablation catheter and the cement injection catheter could be one and the same, so that the bone cement can be injected through a lumen extending through the ablation catheter.

Persons skilled in the art will recognize that the steerable vertebroplasty energy delivery catheter 900 can be used to drill into bones and tissues including soft tissue other than vertebrae and can be used in a variety of surgical applications. In addition, the device can be used for the purpose of cavity creation for the purpose of introducing chemotherapeutic or radiologic agents. The device can also be used for the purposes of mass reduction or elimination of various pathological tissues, e.g., treatment including destruction of cancerous tissue. The disclosures herein should not be construed as limiting the possible medical uses of the steerable vertebroplasty energy delivery catheter 900. In some embodiments, the energy delivery elements of the steerable, curvable catheter can be incorporated with the bone cement delivery elements on one catheter. For example, the ablation steerable, curvable catheter as described in FIGS. 20A-23B could include a closed cement delivery system with an input port such as a Luer lock and one or more cement delivery lumens within the catheter body.

### Steerable, Curvable Cryoablation Catheter for Vertebroplasty

The energy delivery catheter 900 can deliver energy via other techniques than using RF electrodes. For example, the principles of cryoablation can be implemented in place of or in addition to using RF electrodes. Such cryoablation catheters can implement any combination of the features described above. A cryoablation catheter could be used in a variety of applications, including but not limited to ablation of malignant and benign tumors such as, for example, bone, lung, heart, liver, breast, prostate, skin, brain, bladder, uterus, and renal tumors; ablation of ectopic cardiac foci; and alleviation of pain, such as back pain via cryoablation of bony structures, such as a facet or sacroiliac joint, cryoablation, or relief of pain via neurolysis. In some embodiments, the deflectable catheters as described herein can be used for a cryoablation and vertebroplasty (CVT) procedure for cervical, lumbar, thoracic, or sacral primary or metastatic vertebral lesions.

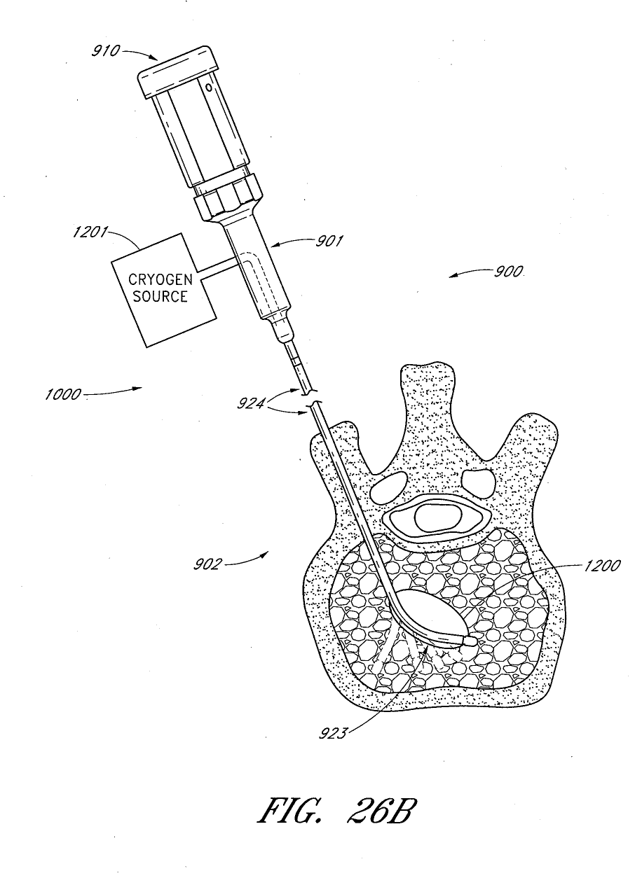

Referring to FIG. 24, one embodiment of a cryoablation catheter is provided. The illustrated cryoablation catheter 1240 can be similar to the energy delivery catheter 900 as illustrated in FIGS. 20A-F. The cryoablation catheter 1240 can include substantially the same steerable, curvable distal end functionalities as described above, for example, in reference to FIGS. 20A-F. However, a cryogenic source 1201 is included in place of an RF generator 1120 and cryotips 1290, 1292 are included as energy delivery elements in place of RF electrodes 1100, 1102. The cryogenic source 1201 can facilitate ablation of tissue, such as cancellous or cortical bone, for example, through a freezing process. The cryogenic source 1201 can bring at least a portion of the cryoablation catheter 1240 to a low temperature. For example, in one embodiment, the cryogenic source 1201 can be used to reduce a temperature of at least a portion of the distal segment 923. Cryogen can cause one or more cryotips to apply extreme cold to tissues to be ablated.

Cryogenic supply source 1201 can provide cryogen to the distal end 1202 of the catheter 1240 through a conduit, e.g., cryogen supply tube 1244. A cryogen connection tube 1242 can provide cryogen from cryogenic supply source 1201 to the cryogenic supply tube 1244.

A variety of substances can be used for cryogen. Examples of such substances include, but are not limited to, liquid nitrogen, helium, argon, hydrogen, oxygen and the like. A cryosurgical system based on liquid nitrogen is manufactured by Cryomedical Sciences, Inc. (Bethesda, Md.). A cryosurgical system based on argon gas is manufactured by EndoCare, Inc. (Irvine, Calif.). In addition, active thawing of an ablation zone (also referred to as an “ice ball”) can be achieved by the infusion of helium gas or the like into cryoprobes configured to cryoablate tissue, instead of the same substance as cryogen used for freezing.

In some embodiments, argon based systems can achieve a more rapid rate of freezing than liquid nitrogen based systems. Additionally or alternatively, argon based systems can provide a more precise control of temperature parameters and time parameters than nitrogen systems. For example, certain argon based systems that include insulated probes and rapid expansion of the gas in the sealed probe tip, can result in rapid cooling that reaches −100° C. within a few seconds. This rapid cooling is sometime referred to as the Joule-Thompson effect.

A cryogen substance can have a default temperature of, for example, less than 0° C., such as, for example, less than about −50° C., −100° C., −150° C., −180° C., −190° C., −200° C., or even less. A thawing substance can have a default temperature of, for example, 20° C. to 80° C., for example, 37° C. to produce immediate thawing. The temperature at each cryotip can be measured separately. In one embodiment, cryoprobes can be actively thawed for 10-15 min. until they reach approximately 25° C., 30° C., 37° C., or more, at which point they can be removed.

As previously noted, cryoablation catheters can include one, two, or more cryotips that can apply extreme cold to tissue to be ablated. For example, the illustrated cryoablation catheter 1240 includes cryotips 1240, 1242. A single cryotip may be sufficient for removing a smaller portion of tissue. For example if a portion of tissue is less, for example 6 cm, 5 cm, 4 cm, 3 cm, 2 cm, 1 cm, or less in dimension, e.g., length, width, diameter, and/or thickness, a single cryotip may be sufficient. In one embodiment, the entire deflectable distal segment 923 can be a cryotip. However, in certain embodiments, more cryotips may be desirable for ablating larger portions of tissue, providing finer control of the ablation process, or for ablating tissue faster. For example, at least 2, 3, 4, 5, 10, 15, 20, 25, 30, or more cryotips may be desirable to ablate portions of tissue having a dimension, e.g., length, width, diameter, and/or thickness larger than 3 cm, 3.5 cm, 4 cm, 5 cm, 6 cm, 8 cm, or 10 cm, or more. In some embodiments, each of the multiple cryotips may be controlled independently. In other embodiments, two or more cryotips may be controlled together. In some embodiments, all cryotip(s) can be included in distal segment 923. In other embodiments, one or more cryotips can be included in distal segment 923 and one or more cryotips can be included in other sections of the cryoablation catheter 1240. The diameter of a single cryoprobe that couples a cryotips can be, for example 1.2-2.4 mm or approximately 11-17 gauge. In some embodiments, each cryotip could extend circumferentially around the outer diameter of the catheter, or partially circumferentially around the outer diameter of the catheter depending on the desired clinical result.

The shaft of a cryoablation catheter can be made of material including, but not limited to, metals, stainless steel, nickel alloys, nickel-titanium alloys, thermoplastics, high performance engineering resins, fluorinated ethylene propylene (FEP), polymer, polyethylene (PE), polypropylene (PP), polyvinylchloride (PVC), polyurethane, polytetrafluoroethylene (PTFE), polyether block amide (PEBA), polyether-ether ketone (PEEK), polyimide, polyamide, polyphenylene sulfide (PPS), polyphenylene oxide (PPO), polysufone, nylon, perfluoro(propyl vinyl ether) (PFA), and combinations thereof.

In some embodiments, a cryo-insulation sheath configured to at least partially cover one, two, or more of the cryotips may be present and made of any suitable material which prevents a cryogenic effect from passing between the shaft and the tissue being treated. For example, the cryo-insulation sheath may be made of vacuum insulation. Other suitable cryo insulating materials include, without limitation, any closed cell foam such as Neoprene®.

The cryogenic supply tube 1244 may be made of any suitable material for delivering cryogen to the distal end of the shaft. In one embodiment, the cryogenic supply tube is made of stainless steel, but any other suitable material may be used. In another embodiment, the cryogenic supply tube can be made of copper.

Cryoablation time may vary from about 15 seconds to about 3½ hours, or longer. In some embodiments, tissue is ablated from 5 to 15 minutes depending upon the size of the region to be ablated. For example, cryoablation can continue for five minutes from the point at which the temperature around the tissue to be ablated is measured, for example, reaches −20° C. or below. In one embodiment, such a temperature can be measured by a temperature sensor. In such ablation, thawing can then be achieved by introducing warm helium gas through the same probes. The ice ball can subsequently melt within, for example, two minutes.

Cryogen can be delivered to a target tissue for any appropriate time period depending on the desired clinical result. For example, in some embodiments tissue to be ablated could be frozen to less than about −10, −25, −40, −50, −60, −70, −80, −90, or −100 degrees Celsius for less than about 20, 15, 10, 8, 5, 2, 1 minutes, 30 seconds, 20 seconds, or less. In some embodiments, tissue can be ablated from 5 to 15 minutes depending upon the size of the region to be ablated. In some embodiments, tissue such as cortical or cancellous bone can be frozen to between about −90 to −110 degrees Celsius for between about 1 to 15 minutes, or between about 5 to 10 minutes.

In one embodiment, a single freeze-thaw cycle can be performed. In other embodiments, two or more freeze-thaw-freeze cycles can be performed for each ablation zone. Each freeze-thaw-freeze cycle can last from 2-60 minutes, for example, it can be from 8-30 minutes. In some embodiments, each part of a freeze-thaw-freeze cycle can have a varying time. For example, a single freeze-thaw-freeze cycle can be preformed with 5-20 minutes for each freeze portion and 2-15 minutes for the thaw portion. In some embodiments, a freeze-thaw-free cycle can be for 10 minutes, 5 minutes, and 10 minutes, respectively. In other embodiments, a freeze-thaw-free cycle can be for 10 minutes, 8 minutes, and 10 minutes, respectively. These times can vary based on, for example, the size of the ablation zone and the number of cryotips.

Referring to FIG. 25, another embodiment of a cryoablation catheter is provided. The illustrated catheter assembly includes a catheter 1250 having a deflectable distal end 1202 and a proximal end 1203. The proximal end 1203 can carry a connecting member 1205, by means of which the catheter is received in a handle 1204. In some embodiments, the catheter 1250 may be for a single use, whereas the handle 1204 is reusable.

To achieve a cooling effect at the distal end 1202 of the catheter 1250, refrigerant is pre-cooled in a cooling apparatus 1225 prior to it being conveyed to a pressure line 1213. The cooling apparatus 1225 can include an isolated cooling chamber 1226, through which a tube 1227 can extend helically. The pressure line 1213 can be connected to the tube 1227. From a source of refrigerant, for example, a pressure cylinder 1228, a pressurized fluid can be supplied to the pressure line 1227. By means of an adjustable valve 1229, a specified quantity of pressurized fluid can be set.

In front of the valve 1229, a line can branch off from the refrigerant line which, via a restriction 1234, can open into the cooling chamber 1226. A quantity of fluid supplied into the cooling chamber 1226 can be set by means of a control valve 1230. When passing a restriction 1234, the refrigerant can expand inside the cooling chamber 1226, and, on doing so, can draw heat from the surroundings. For example, the refrigerant passing through the tube 1227 can consequently be cooled. The expanded fluid can be extracted from the chamber 1226 by a line 1231, so that a sufficient pressure difference is maintained across the restriction.

A temperature sensor 1212 can be arranged at the proximal end of the pressure line 1213 which can be in communication with measuring equipment 1223, for example, via signal line 1211. Thus, a temperature of the refrigerant supplied into the proximal end of the pressure line 1213, can be checked. On the basis of the measured temperature, the control valve 1230 can be set. In another embodiment, the control valve 1230 can be operated by a control apparatus on the basis of the temperature as measured with the sensor 1212.

A temperature sensor (not shown) can also be included near the distal end 1202 of the catheter 1250. This temperature sensor can be in communication with measuring equipment 1223, for example, via signal lines. With the aid of this temperature sensor, the temperature of the distal end 1202 of the catheter can be monitored. In some embodiments, a measured value can also be used to set control valve 1229. In another embodiment, operating the control valve 1229 can be done automatically in accordance with the temperature measured at the distal end 1202 of the catheter.

In some embodiments, at the deflectable distal end 1202 of the catheter 1250, an electrode that could be annular in some embodiments, (not shown) can be provided. This annular electrode can be in communication with measuring equipment 1223, for example, by means of a signal line. In certain embodiments, by means of the annular electrode in combination with an electrically conductive head (not shown), measurements can be taken inside tissues in order to determine the correct position for carrying out the ablation procedure.

The catheter 1250 can include a basic, generally tubular body 1215 with, at the distal end 1202, a closed head made of a thermally conductive material, for instance a metal. The head can include cryoprobe 1290. The generally tubular body 1215 can include one or more lumens 1220 which can serve as a discharge channel.

Inside the lumen 1220, a pressure line 1213 can be received, extending from the proximal end 1203 of the catheter 1250 to the distal end 1202. By means of a bonding agent, the pressure line 1213 can be secured in the head. During the manufacturing process, the distal end of the pressure line 1213 can be first secured in the head, after which the generally tubular body 1215 is pushed over the appropriate section of the head and fixed to it.

The pressure line 1213 can include a restriction at its distal end inside the head. The pressure line 1213 can be led outside the generally tubular body at a Y-piece 1206 in the catheter 1250. The pressure line 1213 and the signal lines can be led outside in the Y-piece 1206 in a sealed manner so that the discharge channel formed by the lumen 1220 remains separate.

Via the pressure line 1213, refrigerant under high pressure can be conveyed to the distal end 1202 of the catheter 1250. After passing the restriction, this refrigerant can expand, drawing heat from the surroundings. Because of this, the head will be cooled to a very low temperature.

The expanded gaseous fluid can return via a discharge channel formed by the lumen 1220, to the proximal end 1203 of the catheter 1250. Inside the handle 1204, the discharge channel can be sealed in an appropriate manner, and can be connected to a line 1232 which discharges the expanded fluid subsequently. A pump 1233 may be received in this line 1232, as is the case in the illustrated example of this embodiment, in order to ensure that also, in case of very small diameters of the catheter 1250, the expanded gas is discharged properly and that a sufficient pressure difference is maintained at the restriction in order to achieve the desired cooling effect.

The pressure line 1213 can be made of a synthetic material having, compared to metal, a low modulus of elasticity and a high thermal resistance coefficient. The catheter 1250, and in particular its distal end 1202, can be made adequately pliable because of the low modulus of elasticity of the material of which the pressure line 1213 has been made. For example, the synthetic material can be any one of many plastics, such as polyamide.

Due to the relatively high thermal resistance coefficient of the material of which the pressure line 1213 can be made, the pre-cooled fluid will typically, at the most, absorb only little heat from the surroundings. Inside the generally tubular body 1215 of the catheter 1250, the pressure line 1213 can extend through the central lumen 1220. The expanded gas which can be discharged from the head passes through lumen 1220. Initially, this expanded gas has a very low temperature and is heated only very slightly in the head. The gas passing through the discharge channel can still have, therefore, a low temperature, so that consequently, also no or only little warming up of the refrigerant supplied under pressure, will occur.

The section of the pressure line 1213 connected to the cooling apparatus 1225 can be provided with an isolation layer in order to prevent warming up of the pressure fluid.

Other elements of cryoablation systems that can be used with the catheter 1240 and/or 1250 described herein can be found, for example, in U.S. Pat. No. 5,807,391 to Wijkamp or U.S. Pat. No. 6,379,348 to Onik, both of which are hereby incorporated by reference in their entirety.

In some embodiments, a steerable cryoablation vertebroplasty catheter can be used to ablate a vertebral body wherein various types of bone cements can be injected as described supra in the application as well as those disclosed in U.S. patent application Ser. No. 11/626,336, filed on Jan. 23, 2007; application Ser. No. 12/029,428 filed on Feb. 11, 2008; application Ser. No. 12/469,654 filed on May 20, 2009; and U.S. Provisional App. No. 61/300,401 filed on Feb. 1, 2010, all of which are hereby incorporated by reference in its entirety. In some embodiments, a steerable, curvable vertebroplasty injector could include one, two, or more cavity creating elements as described in the Ser. No. 12/469,654 application as well as one, two, or more ablation elements as described herein for a multi-function injector, cavity creator, and/or ablation catheter.

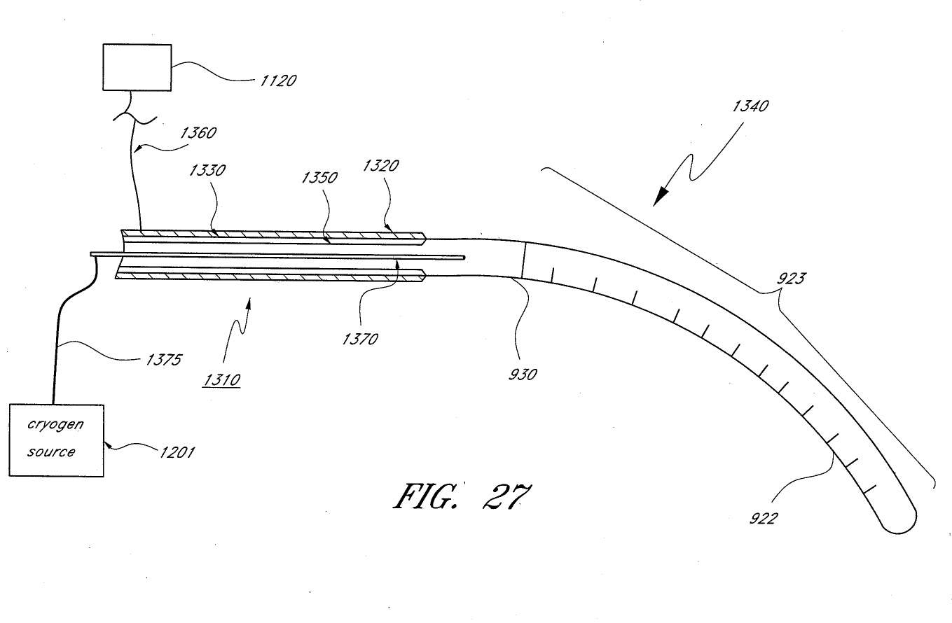

In some embodiments, this procedure for creating a cavity or otherwise preparing the bone for inserting one of the bone cements described above is performed by making an incision in an appropriate location and inserting the distal portion of the steerable cryoablation vertebroplasty catheter to the desired osteotomy site. A number of features of this device minimize the degree of invasiveness required to perform this procedure, which include, but are not limited to, substantially the same features as described above in connection with the steerable energy delivery vertebroplasty catheter 900. One embodiment of a minimally invasive procedure utilizing such features is depicted in FIGS. 26A-B, with schematic ablation zone 1200 illustrated in FIG. 26B.

Referring to FIG. 27, one embodiment of a combined electrosurgical-cryosurgical instrument is provided. The combined electrosurgical-cryosurgical instrument is referred to generally as 1310. Radiofrequency insulation sheath 1320 surrounds the outer surface of shaft 1330, and extends from a proximal end of shaft 1330 to a distal end of shaft 1330, leaving a segment 1340 of the distal end of shaft 1330 radiofrequency-noninsulated. Cryo-insulation sheath 1350 surrounds the inner surface of shaft 1330, and extends from a proximal end of shaft 1330 to a distal end of shaft 1330, leaving a segment 1340 of the distal end of shaft 1330 cryo-noninsulated. RF generator 1120 electrically couples shaft 1330 via cable 1360, and provides electrical energy to segment 1340 of shaft 1330. Cryogenic supply conduit, e.g., tube 1370 within shaft 1330, extends from the proximal end of shaft 1330 to the distal end of shaft 1330. Cryogenic supply source 1201 provides cryogen to the distal end of shaft 1330 through cryogenic supply tube 1370. Cryogen connection tube 1375 provides cryogen from cryogen supply source 1201 to cryogen supply tube 1370.

In operation, combined electrosurgical-cryosurgical instrument 1310 may be inserted into tissue near the site to be ablated. The combined electrosurgical-cryosurgical instrument 1310 can include substantially the same steerable curvable functionalities as the other catheters described herein, including, but not limited to, a distal deflectable segment 923 as previously described.

RF generator 1120 can be used to deliver electrical energy to the distal end of shaft 1330, and cryogen supply source 1201 may be used to provide a cryogenic effect at the distal end of shaft 1330. A tissue can be ablated around the radiofrequency-noninsulated portion 1340 of shaft 1330. Similarly, tissue can be ablated around the cryo-noninsulated portion 1340 of shaft 1330.

Combined electrosurgical-cryosurgical instrument 1310 can also be used to ablate tissue using a combination of RF and cryo techniques described herein. For example, this may be accomplished by supplying the distal end of the shaft with both radiofrequency energy and a cryogenic effect, either sequentially or simultaneously. The resulting ablation may possess the advantages of both radiofrequency ablation and cryoablation. For example, the ablated area may be as large as an area created by certain cryoablation techniques, but not exhibit the toxicity effects that are associated with such cryoablation techniques upon breakdown.

Moreover, the principles and advantages described above in reference to any of the RF ablation or cryoablation catheters can be combined based on at least the concepts disclosed in reference to FIG. 27.

While described herein primarily in the context of vertebroplasty, one of ordinary skill in the art will appreciate that the disclosed energy delivery catheter can be used or modified in a wide range of clinical applications, such as, for example, other orthopedic applications such as kyphoplasty, treatment of any other bones, pulmonary, cardiovascular, gastrointestinal, gynecological, or genitourinary applications. While this invention has been particularly shown and described with references to embodiments thereof, it will be understood by those skilled in the art that various changes in form and details may be made therein without departing from the scope of the invention. For all of the embodiments described above, the steps of the methods need not be performed sequentially. Additionally, the skilled artisan will recognize that any of the above-described methods can be carried out using any appropriate apparatus. Further, the disclosure herein of any particular feature in connection with an embodiment can be used in all other disclosed embodiments set forth herein. Thus, it is intended that the scope of the present invention herein disclosed should not be limited by the particular disclosed embodiments described above.

What is claimed is:

1. A steerable and curvable ablation catheter comprising:
an elongate tubular body having a proximal end and a distal end, wherein the distal end includes a deflectable zone deflectable through an angular range;
a handle on the proximal end of the tubular body;
a deflection control on the handle; and
an ablation element configured to ablate tissue carried by the deflectable zone.

2. The steerable and curvable ablation catheter of claim 1, wherein the ablation element comprises a radiofrequency (RF) electrode.

3. The steerable and curvable ablation catheter of claim 1, wherein the ablation element comprises a cryoprobe.

4. The steerable and curvable ablation catheter of claim 1, further comprising an actuator extending axially between the deflection control and the deflectable zone.

5. The steerable and curvable ablation catheter of claim 1, wherein the actuator comprises an axially moveable element.

6. The steerable and curvable ablation catheter of claim 1, wherein the deflectable zone is in a substantially straight configuration from the proximal end to the distal end in an unstressed state.

7. The steerable and curvable ablation catheter of claim 1, further comprising a plurality of ablation elements.

8. A method of ablating tissue, comprising:
positioning a catheter near a zone of tissue to be ablated, the catheter having an elongate body having a proximal end, a deflection control carried by the proximal end, a deflectable distal end, and an ablation element carried by the deflectable distal end;
deflecting at least a portion of the distal end of the elongate body through an angular range; and
contacting the tissue with the ablation element to ablate the tissue.

9. The method of claim 8, wherein the ablation element is an RF heating electrode.

10. The method of claim 8, wherein the ablation element is a cryoprobe.

11. The method of claim 8, wherein the tissue to be ablated comprises cortical bone.

12. The method of claim 8, wherein the tissue to be ablated comprises cancellous bone.

13. The method of claim 8, wherein the tissue to be ablated comprises a vertebral body.

14. The method of claim 8, wherein the ablation occurs as part of a vertebroplasty procedure.

15. The method of claim 8, wherein the tissue to be ablated comprises a tumor.
